SEQUENCE LISTING (8 sequences)

SEQ ID NO: 1 - protein, 68 aa, Artificial Sequence
  Synthetic- SDF1alpha
KPVSLSYRCPCRFFESHVARANVKHLKILNTPNCALQIVARLKNNNRQVCIDPKLKWIQEYLEKALNK

SEQ ID NO: 2 - protein, 72 aa, Artificial Sequence
  Synthetic- SDF1beta
KPVSLSYRCPCRFFESHVARANVKHLKILNTPNCALQIVARLKNNNRQVCIDPKLKWIQEYLEKALNKRFKM

SEQ ID NO: 3 - protein, 98 aa, Artificial Sequence
  Synthetic- SDF1 fragment
KPVSLSYRCPCRFFESHVARANVKHLKILNTPNCALQIVARLKNNNRQVCIDPKLKWIQEYLEKALNKGRREEKVGKKEKIGKKKRQKKRKAAQKRKN

SEQ ID NO: 4 - protein, 119 aa, Artificial Sequence
  Synthetic- SDF1 fragment
KPVSLSYRCPCRFFESHVARANVKHLKILNTPNCALQIVARLKNNNRQVCIDPKLKWIQEYLEKALNNLISAAPAGKRVIAGARALHPSPPRACPTARALCEIRLWPPPEWSWPSPGDV

SEQ ID NO: 5 - protein, 69 aa, Artificial Sequence
  Synthetic- SDF1 fragment
KPVSLSYRCPCRFFESHVARANVKHLKILNTPNCALQIVARLKNNNRQVCIDPKLKWIQEYLEKALNNC

SEQ ID NO: 6 - protein, 79 aa, Artificial Sequence
  Synthetic- SDF1 fragment
KPVSLSYRCPCRFFESHVARANVKHLKILNTPNCALQIVARLKNNNRQVCIDPKLKWIQEYLEKALNKIWLYGNAETSR

SEQ ID NO: 7 - protein, 67 aa, Artificial Sequence
  Synthetic- shared N-terminal seqeunce of SDF1
KPVSLSYRCPCRFFESHVARANVKHLKILNTPNCALQIVARLKNNNRQVCIDPKLKWIQEYLEKALN

SEQ ID NO: 8 - protein, 21 aa, Artificial Sequence
  Synthetic- shared SDF1 N-terminus 21 amino acid sequence
MNAKVVVVLVLVLTALCLSDG

(19) United States
(12) Patent Application Publication
(10) Pub. No.: US 2024/0044915 A1
(43) Pub. Date: Feb. 8, 2024

(54) USE OF STROMAL CELL-DERIVED FACTOR 1 (SDF1) AS A BIOMARKER FOR DIAGNOSING AND TREATING SEVERE ACUTE RESPIRATORY DISTRESS SYNDROME (ARDS)

(71) Applicant: Ann and Robert H. Lurie Children's Hospital of Chicago, Chicago, IL (US)

(72) Inventor: Youyang Zhao, Deerfield, IL (US)

(21) Appl. No.: 18/258,440

(22) PCT Filed: Dec. 22, 2021
(86) PCT No.: PCT/US21/73066

§ 371 Date: Jun. 20, 2023

Related U.S. Application Data
(60) Provisional application No. 63/129,544, filed on Dec. 22, 2020.

(51) Int. Cl. G01N 33/68; C12N 15/113; A61P 37/04

(52) U.S. Cl. CPC G01N 33/6893; C12N 15/113; A61P 37/04; G01N 2800/125; G01N 2333/521

(57) ABSTRACT

Disclosed herein are methods for diagnosing and treating sepsis, acute respiratory distress syndrome (ARDS), and severe COVID-19 and associated ARDS, and also for treating elderly patients with ARDS induced by sepsis, pneumonia, and COVID-19. The disclosed methods utilize stromal cell-derived factor 1 (SDF1, also known as CXCL12) and the expression level thereof as a biomarker for diagnosing or treating sepsis, ARDS and severe and critical COVID-19.

### CROSS-REFERENCE TO RELATED PATENT APPLICATIONS

This application claims the benefit of priority under 35 U.S.C. § 119(e) to U.S. Provisional Application No. 63/129,544, filed on Dec. 22, 2020, the content of which is incorporated herein by reference in its entirety.

### STATEMENT REGARDING FEDERALLY SPONSORED RESEARCH OR DEVELOPMENT

This invention was made with government support under HL123957 and HL148810 awarded by the National Institutes of Health. The government has certain rights in the invention.

### SEQUENCE LISTING

A Sequence Listing accompanies this application and is submitted as an ASCII text file of the sequence listing named “702581_02075_ST25.txt” which is 6,470 bytes in size and was created on Dec. 9, 2021. The sequence listing is electronically submitted via EFS-Web with the application and is incorporated herein by reference in its entirety.

### BACKGROUND

The field of the invention relates to methods for diagnosing and treating acute respiratory distress syndrome (ARDS), severe COVID-19, and associated ARDS. In particular, the field of the invention relates to the use of stromal cell-derived factor 1 (SDF1) as a biomarker for diagnosing and treating ARDS, for example, ARDS that is induced by sepsis, pneumonia, and COVID-19. The field of the invention also relates to methods for treating ARDS, severe COVID-19 and associated ARDS of elderly patients.

Acute respiratory distress syndrome (ARDS) is a heterogeneous, complex, multi-factorial syndrome with a mortality rate as great as 30-60% (1-3). ARDS can be induced by sepsis, pneumonia, and COVID-19. Endothelial injury characterized by persistently increased lung microvascular permeability resulting in protein-rich lung edema is a hallmark of ARDS. Especially, endothelial injury is the characteristic feature of severe COVID-19 and associated ARDS (4-6). However, the molecular basis of endothelial barrier disruption after sepsis, pneumonia, and COVID-19 remain poorly understood; in particular, little is known about the key molecules and signaling pathways responsible for endothelial barrier dysfunction resulting in high mortality in ARDS patients. Moreover, there is limited knowledge about the molecular mechanisms of endothelial regeneration and vascular repair following inflammatory vascular injury. Especially, the incidence, severity, and mortality of ARDS and COVID-19 are much greater in elderly patients.

Endothelial injury is attributable to disassembly of endothelial adherens junctions (7-9) and endothelial cell (EC) loss resulting from necrosis and programmed cell death including apoptosis, necroptosis, and pyroptosis (10-13). Pyroptosis, distinct from apoptosis and necrosis, is a lytic form of cell death requiring inflammatory Caspases such as Casp4/5/11 (mouse expresses Casp11, the equivalent of human Casp4/5) (11-13) whereas apoptosis, a non-lytic form of cell death is initiated by Casp2, 8, 9 and 10 and requires the effector Casp3, 6, and 7 (14). Pyroptosis occurs most frequently upon infection with intracellular pathogens. Immune cells sensor foreign danger signals within themselves, release pro-inflammatory cytokines such as IL-1β, swell, burst and die. Pyroptosis promotes the rapid clearance of various bacterial and viral infections by removing intracellular replication niches and enhancing host defense (15-18). The role of pyroptosis in sepsis was initially discovered in macrophages by sensing intracellular lipopolysaccharide (LPS) through a TLR4-independent mechanism (12, 13). It has been shown that LPS breaching of the plasma membrane and binding of inflammatory Casp11 or 4/5 activates pyroptosis. Casp11 cleavage of Gasdermin D (GSDMD) releases the active membrane pore-forming GSDMD peptide, which leads to lytic death of cells by swelling (19-21). Thus, under certain pathological conditions, the potentially protective pyroptotic mechanism may activate an exaggerated pathologic response owing to overwhelming cell lysis. A recent study has demonstrated a novel role of pyroptosis in destroying the endothelial barrier through pyroptosis-induced EC lysis, which contributes to the pathogenesis of ARDS (11). However, the aforementioned study fails to define the mechanisms and signaling pathways underlying EC pyroptosis. Crucially, little is known about how extensive EC pyroptosis is activated in septic mice and in ARDS patients.

### SUMMARY

Disclosed herein are methods for diagnosing and treating acute respiratory distress syndrome (ARDS) induced by sepsis and pneumonia, severe COVID-19 and associated ARDS, including methods for diagnosing and treating severe ARDS, such as ARDS that is induced by sepsis, pneumonia, and COVID-19. The disclosed methods utilize stromal cell-derived factor 1 (SDF1, also called CXCL12) and the expression level thereof as a biomarker for diagnosing and treating ARDS and severe COVID-19 and associated ARDS. In a first aspect, methods are disclosed. In some embodiments, the methods comprise: (a) detecting an expression level of stromal cell-derived factor 1 (SDF1) in a biological sample from a subject having or suspected of having acute respiratory distress syndrome (ARDS); and optionally (b) treating the subject for ARDS and severe COVID-19 and associated ARDS. In some embodiments, detecting an expression level comprises detecting a concentration of SDF1 protein in the biological sample. In some embodiments, detecting an expression level comprises detecting a concentration of SDF1 protein in the biological sample, wherein the protein comprises an amino acid sequence selected from the group consisting of SEQ ID NO: 1-7. In some embodiments, the methods further comprise providing a reference concentration of SDF1 and wherein, if the detected concentration of SDF1 protein in the biological sample is equal to or higher than a reference concentration, then the subject is treated for ARDS or severe COVID-19 and associated ARDS. In some embodiments, the biological sample is obtained from the subject at about the time of admission of the subject to intensive care unit (ICU). In some embodiments, the biological sample is obtained from the subject at about the onset of ARDS. In some embodiments, the admission to ICU is due to sepsis, pneumonia, or COVID-19. In some embodiments, the detected plasma concentration and/or reference concentration is at least about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, or higher. In some embodiments, the biological sample is selected from plasma, serum, or blood. In some embodiments, treating the subject comprises administering to the subject an antagonist of C-X-C motif chemokine receptor 4 (CXCR4). In some embodiments, treating the subject comprises administering to the subject a therapeutic agent selected from the group consisting of Plerixafor (AMD3100), CTCE-9908 and their analogs. In some embodiments, the subject is treated for no more than about 3 days. In some embodiments, treating the subject comprises administering to the subject an inhibitor of p110γ phosphoinositide 3-kinase (PI3K) from the group consisting of IPI-549/Eganelisib, Duvelisib, RP6530, RP6503, TG100-115 and Voxtalisib, and their analogs or other p110γPI3k inhibitors or PI3K inhibitors. In some embodiments, treating the subject comprises administering to the subject an siRNA, shRNA, one or more antisense oligos that transiently inhibit expression of CXCR4, or one or more antisense oligos that transiently inhibit expression of p110γPI3K. In some embodiments, the subject is treated for no more than about 3 days. In some embodiments, treating the subject comprises administering to the subject an inhibitor of Caspase 4/5. In some embodiments, treating the subject comprises administering to the subject a therapeutic agent selected from the group consisting of LEVD-fmk and Emricasan and their analogs. In some embodiments, treating the subject comprises administering to the subject an siRNA, shRNA, an antisense oligo, a CRISPR/guide RNA, or a genome editing system that inhibits expression of Caspase 4/5, or a dominant negative Caspase 4/5 that inhibits Caspase 4/5 function. In some embodiments, treating the subject comprises administering to the subject an inhibitor of gasdermin D (GSDMD) or gasdermin E (GSDME). In some embodiments, treating the subject comprises administering to the subject disulfiram optionally with copper gluconate, or disulfiram analogs. In some embodiments, treating the subject comprises administering to the subject an siRNA, shRNA, an antisense oligo, a CRISPR/guide RNA, or a genome editing system that inhibits expression of GSDMD or GSDME. In some embodiments, treating the subject comprises administering to the subject one or both of (1) a dominant negative GSDMD that inhibits GSDMD function, and (2) a dominant negative GSDME that inhibits GSDME function. In some embodiments, the methods comprise diagnosing the subject having acute respiratory distress syndrome (ARDS) or severe COVID-19 and associated ARDS for treatment 1, 2, 3, 4, 5, or 7 days after ICU admission. In some embodiments, the methods comprise detecting an expression level of stromal cell-derived factor 1 (SDF1) in a biological sample (blood, plasma, serum, lung biopsy) from a subject having ARDS, or severe COVID-19 and associated with ARDS in a series of days following ICU admission (day 0), e.g. day 1, day 2, day 3, day 4, day 5 and day 7. In some embodiments, if no marked increases (at least 50%) of SDF1 levels in any one or more days from day 2 to day 7 compared to day 0 or day 1 are observed, the subject will be treated with SDF1 or activators of SDF1 at day 3 or after. In some embodiments, treating the subject comprises administering to the subject a recombinant human SDF1. In some embodiments, the recombinant human SDF1 comprises a protein with the amino acid sequence of any of SEQ ID NOs: 1-7, Ser-SDF1(S4V), or their analogues. In some embodiments, treating the subject comprises administering to the subject a SDF1 small peptide from the group of CTCE-0214, CTCE-0324, and their analogs. In some embodiments, treating the subject comprises administering to the subject SDF1 comprising a protein comprising the amino acid sequence of any SEQ ID NOs:1-7, Ser-SDF1(S4V), or their analogs using a viral vector, a non-viral vector, cell, stem cells, mesenchymal stem cells, or microvesicles from the cells as a carrier. In some embodiments, the non-viral vector is a nanoparticle or a liposome. In some embodiments, treating the subject comprises administering to the subject a CXCR4 activator. In some embodiments, treating the subject comprises administering to the subject a p110gamma PI3K activator. In some embodiments, the subject is an elderly patient, e.g., at age of 65, 70, 75, 80 years or older.

In another aspect of the current disclosure, further methods are disclosed. In some embodiments, the methods comprise administering treatment for acute respiratory distress syndrome (ARDS) or severe COVID-19 to a subject exhibiting a concentration of stromal cell-derived factor 1 (SDF1) protein in the blood (plasma, serum) sample from the subject at admission to ICU or early onset of ARDS that is at least about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, or higher. In some embodiments, the treatment comprises administering to the subject an antagonist of C-X-C motif chemokine receptor 4 (CXCR4). In some embodiments, the treatment comprises administering to the subject a therapeutic agent selected from the group consisting of Plerixafor (AMD3100) and CTCE-9908 and their analogs. In some embodiments, the treatment comprises administering to the subject an inhibitor of p110γ phosphoinositide 3-kinase (PI3K). In some embodiments, the treatment comprises administering to the subject a therapeutic agent selected from the group consisting of IPI-549/Eganelisib, Duvelisib, RP6530, RP6503, TG100-115 and Voxtalisib and their analogs, or other p110γ PI3k inhibitors, or PI3K inhibitors. In some embodiments, treating the subject comprises administering to the subject an siRNA, shRNA, one or more antisense oligos that transiently inhibit expression of CXCR4 or one or more antisense oligos that transiently inhibit expression of p110γ PI3K. In some embodiments, the treatment comprises administering to the subject an inhibitor of Caspase 4/5. In some embodiments, the treatment comprises administering to the subject a therapeutic agent selected from the group consisting of LEVD-fmk and Emricasan and their analogs. In some embodiments, the treatment comprises administering to the subject a siRNA, shRNA, an antisense oligo, a CRISPR/guide RNA, a genome editing system that inhibits expression of Caspase 4/5, or a dominant-negative Caspase 4/5 that inhibits Caspase 4/5 function. In some embodiments, the treatment comprises administering to the subject an inhibitor of gasdermin D (GSDMD) or gasdermin E (GSDME). In some embodiments, the treatment comprises administering to the subject disulfiram optionally with copper gluconate, or disulfiram analogs. In some embodiments, the treatment comprises administering to the subject a siRNA, shRNA, an antisense oligo, a CRISPR/guide RNA, or a genome editing system that inhibits expression of GSDMD or GSMDE. In some embodiments, treating the subject comprises administering to the subject a dominant negative GSDMD that inhibits GSDMD function. In some embodiments, the subject is an elderly patient, e.g., at age of 65, 70, 75, 80 years or older.

### BRIEF DESCRIPTION OF THE FIGURES

FIG. 1. ELISA quantification demonstrating that high levels of plasma SDF1 at admission are associated with greater mortality of ARDS patients. Plasma and BALF were collected from ARDS patients at the day of admission. There was no significant difference in age between the 2 ARDS groups. Plasma and BALF were also collected from critically ill non-ARDS (control) subjects. ELISA was carried out to quantify SDF1 levels in plasma (A) and BALF (B). Mortality of ARDS patients was recorded within 14 days after diagnosis. Bar=mean. Student's t test.

FIG. 2. Recombinant (r)SDF1 priming augmented LPS-induced lung injury and mortality in mice. WT mice treated with either vehicle (PBS) or rSDF1 (4 ug/kg, i.v., 2 injections) under 3 protocols—8 h & 0 h (Pre=pretreatment), 0 h & +6 h (Ccm=concomitant), or +2 h & +8 h (Post=post-treatment) were challenged with LPS (2.5 mg/kg, i.p.). At 16 h post-LPS, lung tissues were collected for EBA extravasation assay (A), MPO activity assay (B). *P<0.05; **, P<0.01, ***, P<0.001 compared to LPS alone. ††, P<0.01; †††, P<0.001. One-way ANOVA with a Games-Howell post hoc analysis for multiple group comparisons. Bar=mean. (C) Mortality study. Mice treated with either rSDF1 or PBS were challenged with LPS (6 mg/kg, i.p.). Mortality were monitored for 96 h. ***, P<0.001 compared to LPS only. Log-rank (Mantel-Cox) test, n=5mice/group. Pretreatment of rSDF1 resulted in a marked increase of LPS-induced mortality than concomitant treatment. f, P<0.05. (D) Representative H & E staining showing rSDF1 pretreatment augmented LPS (2.5 mpk)-induced inflammatory lung injury evident by increased neutrophil infiltration, septal thickening, and hemorrhaging. Scale bar, 100 μm.

FIG. 3. Pre-expression of SDF1 in lung ECs augmented LPS-induced lung injury and mortality. WT mice were transduced with mixture of liposome: SDF1 plasmid (SDF1) or empty Vector (Vec) and 20 h later when SDF1 was expressed (A, freshly isolated ECs and non-ECs), challenged with LPS (2.5 mg/kg. i.p.). At 16 h post-LPS, lung tissues were collected for EBA flux (B), wet/dry weight ratio (C), and MPO activity (D). Bar=mean, ***, P<0.001. (E) Mortality at 20 h post-liposome:DNA (Vector or SDF1 plasmid), the mice were challenged with LPS (6 mg/kg, i.p.) and mortality were monitored for 96 h. ***, P<0.001. n=10mice/group.

FIG. 4. Endothelial Cxcr4 mediates SDF1 priming-augmented lung injury and mortality following LPS challenge. (A) Model of tamoxifen-inducible EC-restricted deletion of Cxcr4 (iΔEC). Quantitative RT-PCR demonstrating tamoxifen treatment-induced Cxcr4 deletion in ECs (CD45⁻/CD31⁺) but not in leukocytes (CD45⁺ cells). (B-D) SDF1 priming-augmented LPS-induced lung injury (2.5 mg/kg, i.p., 16 h) was inhibited in Cxcr4^{iΔEC} mice evident by decreased EBA flux (B), Wet/Dry weight ratio (C), and MPO activity (D) compared to WT mice. **, P<0.01, ***, P<0.001. (E) Marked increase of survival of Cxcr4^{iΔEC} mice challenged with SDF1/LPS. ***, P<0.001.

FIG. 5. p110γPI3K mediates SDF1 priming-augmented lung injury and mortality following LPS challenge. (A-C) SDF1 priming (liposome:SDF1 plasmid)-augmented lung injury induced by LPS (2.5 mg/kg, i.p., 16 h) was inhibited in Pi3kcg^{-/-} (KO) mice evident by decreased EBA flux (A), Wet/Dry weight ratio (B), and MPO activity (C) compared to WT mice. (D) Marked increase of survival of Pi3kcg^{-/-} (KO) mice challenged with SDF1/LPS (6 mg/kg, i.p.). *, P<0.05, ***, P<0.001.

FIG. 6. Restoration of endothelial p110γ expression in Pik3cg^{-/-} lungs normalized SDF1 priming effects following LPS challenge. (A) EBA extravasation assay showing increased vascular leak in Pik3cg^{-/-} (KO) lungs expressing endothelial p110γPI3K and SDF1 as seen in WT with Vec+SDF1 plasmids at 16 h post-LPS. (B) Restored inflammatory response in Pik3cg^{-/-} lungs expressing endothelial p110γPI3K and SDF1 following LPS challenge. **, P<0.01.

FIG. 7. Marked increases of EC death in SDF1 or Casp11 plasmid-transduced mice following LPS challenge. (A) Representative flow cytometry histograms demonstrating increased EC death in SDF1 or Casp11 plasmid-transduced mice compared to vector-transduced mice following LPS challenge (2.5 mg/kg, i.p.). At 16 h post-LPS (or PBS), lung cells were isolated for Casp1-FILCA 660 staining which bound only the active form of Casp1. Nuclei were stained with DAPI for FACS analysis of cell death. GFP⁺ cells (ECs) were gated for analysis. (B) Quantification of Casp1-independent EC death. CTL=no liposome; Lipo=liposome; Vec=liposome+Vector, SDF1=liposome+SDF1 plasmid, C11=liposome+Casp11 plasmid. ***, P<0.001.

FIG. 8. SDF1 priming drastically increased Gsdmd expression and cleavage in mouse lung ECs but not non-ECs. After 8 h rSDF1 priming, the mice were challenged with LPS and lung tissues at 16 h post-LPS were collected for EC (CD45⁻ CD31⁺) and non-EC (CD45⁻/CD31⁻) isolation. Western blotting was carried out with anti-Gsdmd. Anti-β-actin was used as loading control. N-Gsdmd=Gsdmd cleavage.

FIG. 9. Casp11 mediates SDF1 priming effects in LPS-challenged mice. WT or Casp11^{-/-} mice were primed with rSDF1 (SDF) or vehicle (PBS) and then challenged with LPS (2.5 mg/kg, i.p.). At 16 h post-LPS, lung tissues were collected for EBA extravasation (A) and MPO activity assay (B). **, P<0.01. (C) After SDF1 priming, WT and Casp11^{-/-} mice were challenged with LPS (6 mg/kg, i.p.), and mortality were recorded for 48 h. ***, P<0.001 (n=10 mice/group).

FIG. 10. Marked increase of Casp11 expression in lung ECs but not non-ECs in SDF1/LPS-treated mice. QRT-PCR analysis of expression of Casp11 (A), Casp1 (B), and Casp8 (C) in freshly isolated lung ECs and non-ECs. At 16 h post-LPS (2.5 mg/kg, i.p.), ECs and non-ECs were isolated from naïve (CTL), rSDF1 only (SDF1), LPS only (LPS), and rSDF1 primed/LPS (SDF/LPS)-treated mice for RNA isolation and QRT-PCR analysis. ***, P<0.001. Bars represent mean.

FIG. 11. Marked increases of expression of CD14 and LBP in lung ECs but not non-ECs in SDF1/LPS-treated mice. At 16 h post-LPS (2.5 mg/kg, i.p.), ECs and non-ECs were freshly isolated from naïve (Control), SDF1 only (SDF1), LPS only (LPS), and SDF1/LPS-treated mice for RNA isolation and QRT-PCR analysis. ***, P<0.001 versus ECs-Control. Bars represent mean.

FIG. 12. Marked increases of LPS binding in lung ECs from rSDF1-primed mice compared to PBS-treated mice. After rSDF1 (or PBS) priming, WT mice were challenged with LPS. At 16 h post-LPS, mouse lungs were collected for cell isolation followed by incubation with LPS-AF488 (1 ug/ml) for 30 min on ice. The cells were then immunostained with anti-CD45 and anti-CD31. CD45⁻ cells were gated for analysis. (A) representative diagrams of FACS analysis. (B) Quantification of LPS⁺ ECs (CD45⁻/CD31⁺ cells). (C) Quantification of LPS^{hi} ECs. ***, P<0.001.

FIG. 13. Loss of endothelial SDF1 impairs vascular repair and increases mortality following LPS challenge. (A) Marked induction of endothelial SDF1 expression after LPS challenge. At various times after LPS challenge (2.5 mg/kg, i.p.), lung tissues were collected from WT and Cxc112^{Tie2Cre} (CKO) mice for quantitative RT-PCR analysis. SDF1 was induced in late injury phase in WT lungs whereas almost blunted in CKO lungs, indicating SDF1 was mainly induced in lung ECs. (B) Quantitative RT-PCR analysis showing EC-specific disruption of SDF1 expression in CKO mice. (C) Impaired vascular repair in CKO lungs after LPS challenge. Vascular permeability was similarly increased in both WT and CKO mice during the injury phase (36 h). (D) Increased mortality in CKO mice after LPS challenge (4 mg/kg, i.p.). *, P<0.05. **, P<0.01. ***, P<0.001.

FIG. 14. Impaired resolution of inflammation in CKO lungs after LPS challenge. (A) Time course of MPO activity in mouse lungs after LPS challenge. (B, C) Quantitative RT-PCR analysis demonstrating persistent increases of proinflammatory gene expression. (D) Representative micrographs of H & E staining showing persistent inflammatory lung injury in CKO mice at 72 h post-LPS including leukocyte sequestration and septal thickening. *, P<0.05. ** P<0.01.

FIG. 15. Impaired vascular repair in aged WT mice is ascribed to failure in SDF1 induction. (A) Failed induction of SDF1 expression in aged (22 months old) mice following LPS challenge in contrast to young adult mice (3-4 months old). (B-D) Forced expression of SDF1 in lung ECs restored vascular repair and resolved inflammation in aged mice following LPS challenge. 22 months old WT mice were challenged with LPS (0.5 mg/kg, i.p.) and then administrated (i.v.) with mixture of liposome:plasmid DNA expressing human SDF1 under the control of CDH5 promoter (SDF1) or empty vector (Vector) at 16 h post-LPS. At 72 h post-LPS, lung tissues were collected for EBA flux assay (B), MPO activity analysis (C) and H & E histology (D). (E) Forced expression of SDF1 in lung ECs promoted survival in aged mice following LPS challenge (1.5 mg/kg, i.p.). **, P<0.01. ***, P<0.001.

FIG. 16. Increased endothelial cell proliferation in lungs of aged mice with forced expression of SDF1 after LPS challenge. (A) representative micrographs of anti-BrdU staining showing reactivation of lung EC proliferation at 72 h post-LPS challenge in aged mice transduced with mixture of liposome:SDF1 plasmid DNA. Arrows indicate BrdU-positive ECs. (B) Quantification of cell proliferation in lungs of aged mice at 72 h post-LPS. ***, P<0.001 versus Vector.

FIG. 17. Impaired lung vascular repair seen in aged WT mice was rescued by forced expression of SDF1 in lung ECs following polymicrobial sepsis challenge. (A) Quantitative RT-PCR analysis demonstrating failed induction of SDF1 in lungs of aged mice in contrast to young adult mice. (B, C) Forced SDF1 expression normalized lung vascular repair and resolution of inflammation evident by normal vascular permeability (B) and neutrophil sequestration (C) at 96 h post-polymicrobial sepsis induced by cecal ligation and puncture (CLP). At 16 h post-CLP, liposome:plasmid DNA were administered i.v. and lung tissues were collected at 96 h post-CLP. (D) Forced expression of SDF1 promoted survival of aged mice following CLP challenge. **, P<0.01.

FIG. 18. Therapeutic treatment with recombinant SDF1 (rSDF1) normalized vascular repair in lungs of aged mice following LPS challenge. Aged WT mice (22 months old) were challenged with LPS (0.5 mg/kg, i.p.). At 24 h post-LPS, the mice were treated with either rSDF1 (i.v.) or vehicle (PBS) and at 72 h post-LPS, lung tissues were collected for assessment of EBA flux (A), wet/dry weight ratio (indicator of lung edema) (B) and MPO activity (C). **, P<0.01. ***, P<0.001.

### DETAILED DESCRIPTION

The present invention is described herein using several definitions, as set forth below and throughout the application.

As used in this specification and the claims, the singular forms “a,” “an,” and “the” include plural forms unless the context clearly dictates otherwise. For example, the term “a biomarker” and a “therapeutic agent” should be interpreted to mean “one or more biomarkers” and “one or more therapeutic agents,” respectively, unless the context clearly dictates otherwise. As used herein, the term “plurality” means “two or more.”

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

As used herein, the term “subject” may be used interchangeably with the term “patient” or “individual” and may include an “animal” and in particular a “mammal.” Mammalian subjects may include humans and other primates, domestic animals, farm animals, and companion animals such as dogs, cats, guinea pigs, rabbits, rats, mice, horses, cattle, cows, and the like.

As used herein, a “subject in need thereof” includes a subject having or at risk for developing acute respiratory distress syndrome (ARDS), and particularly severe ARDS. A subject in need thereof may include a subject that has or is at risk for developing ARDS, particularly severe ARDS, which has been induced by sepsis, pneumonia, and/or COVID-19 disease. A subject in need thereof may, in some embodiments, not include a subject that is suffering from sterile sepsis as such conditions may not result in induction of pyroptosis. A subject in need thereof may include a subject that is exhibiting a relatively high concentration level of stromal cell-derived factor 1 (SDF1). For example, a subject in need thereof may include a subject that is exhibiting a concentration of SDF1 in plasma of the subject or in lungs of the subject (or in a biological sample from the subject comprising plasma) that is at least about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, or higher.

As used herein a “subject sample” or a “biological sample” from the subject refers to a sample taken from the subject, such as, but not limited to a fluid sample (e.g., plasma, serum, blood, bronchioalveolar fluid (e.g., as obtained by performing a bronchioalveolar lavage), saliva, urine, stool, cerebrospinal fluid, etc.) or a tissue sample (e.g, lung, alveoli, etc.).

As used herein, the term “biomarker” refers to a characteristic that is objectively measured and evaluated as an indicator of normal biological processes, pathogenic processes, or pharmacological responses to a therapeutic intervention. By way of example but not by way of limitation, biomarkers disclosed herein include products of gene expression such as RNA and/or protein. In some embodiments, an aberrant level of gene expression (e.g., an increased or decreased level of expression in a biological sample) as compared to a control, threshold, or baseline level of expression is indicative of susceptibility or resistance to a disease state (e.g., severe ARDS).

Exemplary genes that serve as biomarkers for use in the disclosed methods may include stromal cell-derived factor 1 (SDF1). The gene for SDF1 (also referred to as C-X-C motif chemokine 12 (CXCL12) is present on human chromosome 10 at the band 10q11.1 and contains 9 exons. The gene expresses 6 isoforms of SDF1 through alternative splicing. All the SDF1 precursor proteins share an N-terminus sequence of 21 amino acids, mnakvvvvlvlvltalclsdg (SEQ ID NO:8). The expressed isoforms share a N-terminal sequence of 67 amino acids but have distinct C-termini (i.e., a.a 68→) as follows:

| (SDF1α) | | |
|---|---|---|
| SEQ ID NO: 1 | | |
| 1 | kpvslsyrcp crffeshvar anvkhlkiln tpncalqiva rlknnnrqvc idpklkwiqe ylekaln k | |
| (SDF1β) | | |
| SEQ ID NO: 2 | | |
| 1 | kpvslsyrcp crffeshvar anvkhlkiln tpncalqiva rlknnnrqvc idpklkwiqe ylekaln | |
| | krfkm | 72 |
| SEQ ID NO: 3 | | |
| 1 | kpvslsyrcp crffeshvar anvkhlkiln tpncalqiva rlknnnrqvc idpklkwige ylekaln | |
| | kgrreekvgk kekigkkkrq kkrkaaqkrk n | |
| SEQ ID NO: 4 | | |
| 1 | kpvslsyrcp crffeshvar anvkhlkiln tpncalqiva rlknnnrqvc idpklkwiqe ylekaln | |
| | nlisaapagk rviagaralh psppracpta ralceirlwp ppewswpspg dv | |
| SEQ ID NO: 5 | | |
| 1 | kpvslsyrcp crffeshvar anvkhlkiln tpncalqiva rlknnnrqvc idpklkwiqe ylekaln nc | |
| SEQ ID NO: 6 | | |
| 1 | kpvslsyrcp crffeshvar anvkhlkiln tpncalqiva rlknnnrqvc idpklkwiqe ylekaln | |
| | kiwlygnaet sr | |
| The shared N-terminal sequence of 67 amino acids of the SDF1 has a sequence as | | |
| follows: | | |
| SEQ ID NO: 7 | | |
| 1 | kpvslsyrcp crffeshvar anvkhlkiln tpncalqiva rlknnnrqvc idpklkwige ylekaln | 67 |

As such, any and all of the different isoforms may be detected using an antibody or antigen-binding fragment thereof that recognizes and binds to an epitope present in this shared 88 amino acid sequence, e.g., SEQ ID NO:8 and SEQ ID NO: 7). In the methods disclosed herein, SDF1, including any or all of its isoforms, may be detected by contacting a biological sample with an antibody or antigen-binding fragment thereof that recognizes and binds to an epitope present in SEQ ID NO:8 and forms a complex with SDF1 and detecting the complex. The antibody or antigen-binding fragment thereof may be labeled (e.g., with a fluorescent label or radiolabel or enzyme), and/or the disclosed methods may utilize a secondary antibody that binds the complex, where the secondary antibody is labeled (e.g., with a fluorescent label or radiolabel or enzyme).

The disclosed methods may include detecting the protein concentration of a biomarker in a biological sample. Suitable methods for detecting the protein concentration of a biomarker in a biological sample may include one or more techniques selected from the group consisting of: immunoassays assays, such as ELISA and Western blotting; chromatographic methods; and protein mass spectrometry assays. Antibodies that bind to specific proteins are well-known in the art and some are commercially available, as are ELISA kits.

The disclosed methods may include detecting the mRNA concentration of a biomarker in a biological sample, e.g., sputum, lavage fluid, bronchioalveolar lavage fluid, a biopsy sample, e.g., a lung biopsy. Methods to detect RNA are well known in the art, and numerous kits and options are commercially available. By way of example, but not by way of limitation, methods include reverse transcription and polymerase chain reaction, (RT-PCR), and methods employing direct oligonucleotide probe hybridization to the biomaker RNA e.g., Northern blotting. In the disclosed methods, the mRNA that is detected may include mRNA encoding an amino acid sequence of any of SEQ ID NOs:1-7.

As used herein, the term control sample, control level, reference concentration, or control subject, refer to a sample, level, or subject that is considered “normal” or “wild-type” relative to the specific condition or conditions under investigation. For example, a biomarker control level is the level of the biomarker identified in a subject or a cohort of subjects (e.g., pooled samples, or averaged values) that are not symptomatic for, and have no known precondition for developing a disease or condition in question (e.g., ARDS and in particular severe ARDS). The levels are compared and a threshold level or a baseline level is determined: expression levels below the threshold or baseline level are considered “low” and are therefore indicative of normal expression level of, for example, SDF1; expression levels above the threshold or baseline level are considered “elevated” and are therefore indicative of susceptibility to a therapeutic drug. In some embodiments, elevated levels of a biomarker, e.g., SDF1, are associated with an increase in mortality in subjects. In particular, elevated levels of the biomarker SDF1, especially when the level is measured at the time of admission of the subject to an intensive care unit, or other similar unit, (e.g., intensive therapy, intensive treatment, or critical care, where subjects with severe or life-threatening illnesses and injuries are treated), or when measured at the time of diagnosis of ARDS, are associated with increased mortality. Therefore, in some embodiments, such subjects are considered good candidates for treatment with the novel methods disclosed herein.

In some embodiments, a subject's biomarker levels can be determined before, during, and/or after a course of treatment or therapy, or throughout the subject's life, e.g., if a genetic predisposition exists or if clinical symptoms regularly appear. In some embodiments, after a subject's biological samples has been identified as having elevated biomarker levels, the subject may be assessed with further diagnostic methods and/or treatment methods for ARDS, and in particular severe ARDS.

### Use of Stromal Cell-Derived Factor 1 (SDF1) as a Biomarker for Diagnosing and Treating Acute Respiratory Distress Syndrome (ARDS).

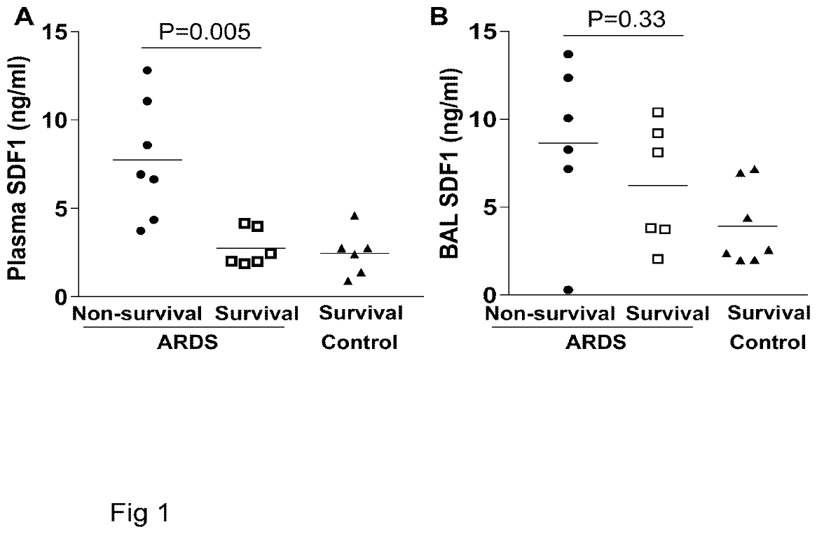

The instant disclosure demonstrates that stromal cell-derived factor 1 (SDF1, also called CXCL12) priming is a novel and critical activator of EC pyroptosis leading to severe lung injury and greater mortality in mice following lipopolysaccharide (LPS) challenge. This finding is consistent with the fundamental observation in ARDS patients that elevated plasma SDF1 levels at diagnosis is associated with greater mortality (FIG. 1). The disclosed mechanistic studies demonstrate that SDF1 priming induces expression of Casp11 and LPS co-receptors CD14 and LBP selectively in ECs, leading to increased gasmodermin D (GSDMD) cleavage (i.e. activation) in ECs and thereby activation of overwhelming EC pyroptosis. The inventor has also delineated the signaling pathways leading to SDF1-induced pyroptosis and endothelial injury. As used herein, “pyroptosis” refers to a highly inflammatory form of lytic programmed cell death that occurs most frequently upon infection with intracellular pathogens. Identifying SDF1 as the critical activator of EC pyroptosis provides not only a novel and important prognostic biomarker of severity and mortality of ARDS patients, but also novel druggable targets, which are essential for the development of novel therapeutic approaches to inhibit this endothelial injury program to prevent injury in and promote survival of ARDS patients in a subpopulation of patients with high risk of severe injury and great mortality owing to high levels of plasma SDF1 at the early onset of the disease. Thus, the instant disclosure provides targeted therapies for the treatment of such diseases and disorders.

Furthermore, the disclosed studies using a novel mouse model with EC-restricted disruption of SDF1 have shown the critical role of endothelial SDF1 induced after sepsis challenge in mediating endothelial repair. Also observed was failed induction of endothelial SDF1 expression in aged lungs after sepsis challenge and associated defective vascular repair and high mortality and forced expression of SDF1 reactivated endothelial regeneration and vascular repair and promoted survival in aged mice. Treatment of recombinant human (rh) SDF1 resulted in activation of vascular repair and resolution of inflammation in aged mice. Thus, rhSDF1 or SDF1 mimetics are potential therapeutics for treatment of severe ARDS with defective induction of SDF1 after injury, such as in elderly ARDS patients.

The methods disclosed herein relate to methods of diagnosing and treating acute respiratory distress syndrome (ARDS), such as ARDS that is induced by, for example, sepsis, pneumonia, and COVID-19. The methods disclosed herein typically utilize stromal cell-derived factor 1 (SDF1, also known as CXCL12) as a biomarker for diagnosing and treating ARDS. In some embodiments, the plasma SDF1 level at admission to the Intensive Care Units (ICU) (day 0) or early onset of ARDS (day 1) is used as a biomarker to guide treatment. In some embodiments, the SDF1 levels in biological samples (e.g., plasma) at various days (day 0, 1, 3, 5, etc.) after admission to ICU are used as a biomarker to guide treatment of ARDS.

In some embodiments of the disclosed methods, the subject is elderly, e.g., age 65, 66, 67, 68, 69, 70, 71, 72, 73, 74, 75, 76, 77, 78, 79, 80, 81, 82, 83, 84, 85, 86, 88, 89, 90, 91, 92, 93, 94, 95, 96, 97, 98, 99, 100 years, or older. In some embodiments, the elderly subject has been diagnosed with ARDS.

In some embodiments, the disclosed methods may comprise: (a) detecting an expression level of stromal cell-derived factor 1 (SDF1) in a biological sample from a subject having or suspected of having acute respiratory distress syndrome (ARDS); and, optionally, (b) treating the subject for ARDS. Expression levels of SDF1 may be detected by detecting a concentration of SDF1 protein in the biological sample (e.g., detecting a concentration of a protein comprising the amino acid sequence of any of SEQ ID NOs:1-7 in the biological sample). Expression levels of SDF1 also may be detected by detecting a concentration of SDF1 mRNA in the biological sample (e.g., detecting a concentration of an mRNA encoding the amino acid sequence of any of SEQ ID NOs:1-7 in the biological sample).

In some embodiments, the disclosed methods include detecting a concentration of SDF1 protein in the biological sample (e.g., detecting a concentration of a protein comprising the amino acid sequence of any of SEQ ID NOs:1-8 in the plasma sample). In further embodiments, if the detected concentration of SDF1 protein in the biological (e.g. plasma) sample at admission to ICU or at the early onset of ARDS is equal to or higher than a reference concentration, then the subject is treated to prevent ARDS or inhibit ARDS progression. In even further embodiments, if the detected concentration and/or reference concentration is at least about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, or higher, then the subject is treated for ARDS, in particular severe ARDS.

The novel methods of using SDF1 as a biomarker for diagnosing and treating ARDS stem from the inventor's discovery that the level of SDF1 in biological samples from subjects at the time of admission, or early onset of ARDS is correlated with increased severity, e.g., mortality in the subjects. Therefore, timely collection of biological samples aids in establishing a timeline for SDF1 levels that may be used, in the novel methods disclosed herein, to guide treatment of ARDS and improve subject outcomes.

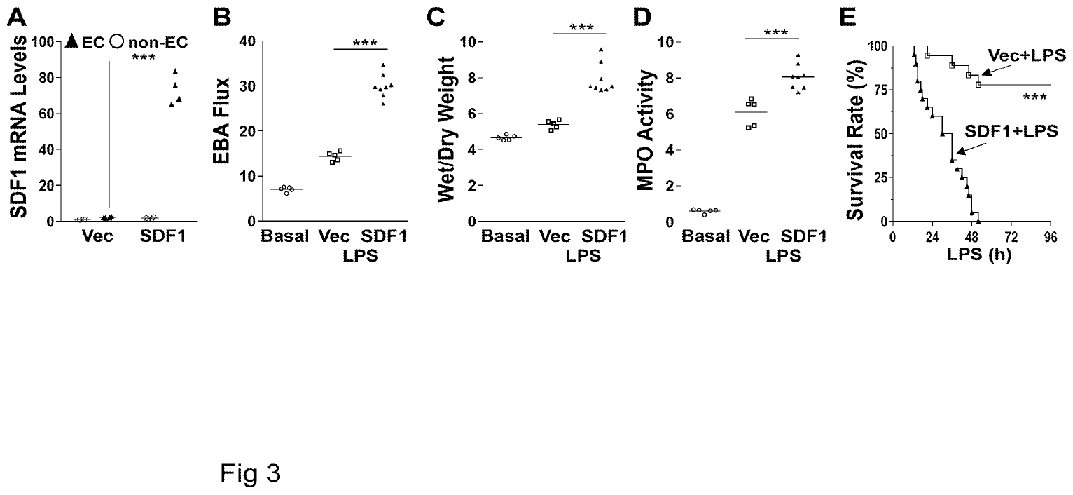

Suitable biological samples for the disclosed methods may include biological samples that are isolated from the subject such as blood or blood products (e.g., plasma) and/or lung fluid (e.g., bronchoalveolar lavage fluid (BALF)). Suitable biological samples may include tissue biopsies (e.g., lung biopsies). Without being limited by any theory or mechanism, the inventors have demonstrated that SDF1 expression primes endothelial cells (ECs) in the lungs to undergo pyroptosis when faced with certain immunological challenges, e.g., sepsis, pneumonia, COVID-19, etc. See, for example, FIG. 3, which demonstrates that expression of SDF1 in endothelial cells increased mortality after challenge with lipopolysaccharide (LPS), a model system for studying endothelial injury.

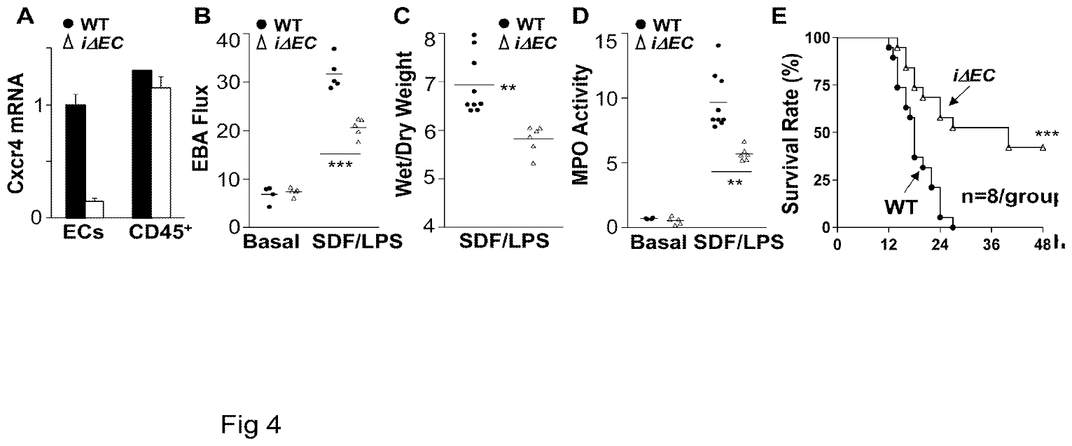

The disclosed methods describe targeted therapy using plasma SDF1 levels at the time of admission to ICU or early onset of ARDS as a biomarker. Previously, researchers have demonstrated that both CXCR4 and CXCR7 can act as receptors for SDF1. However, the inventor demonstrated that knockout of CXCR4 reduced mortality in mice challenged with a lethal dose of LPS. See, for example, FIG. 4. Therefore, in some embodiments, the subject with elevated plasma SDF1 level at admission to ICU or early onset of ARDS is treated by administering to the subject an antagonist of C-X-C motif chemokine receptor 4 (CXCR4). Suitable antagonists of CXCR4 may include, but are not limited to, Plerixafor (AMD3100) and its analogs, BL-8040, CXCR4 inhibitor, CXCR4 antibody, SDF1 inhibiting peptides which include but not limited to CTCE-9908, and its analogs. In addition, other methods of inhibiting CXCR4 include, e.g., administering to a subject one or more antisense oligos that transiently inhibit expression of CXCR4.

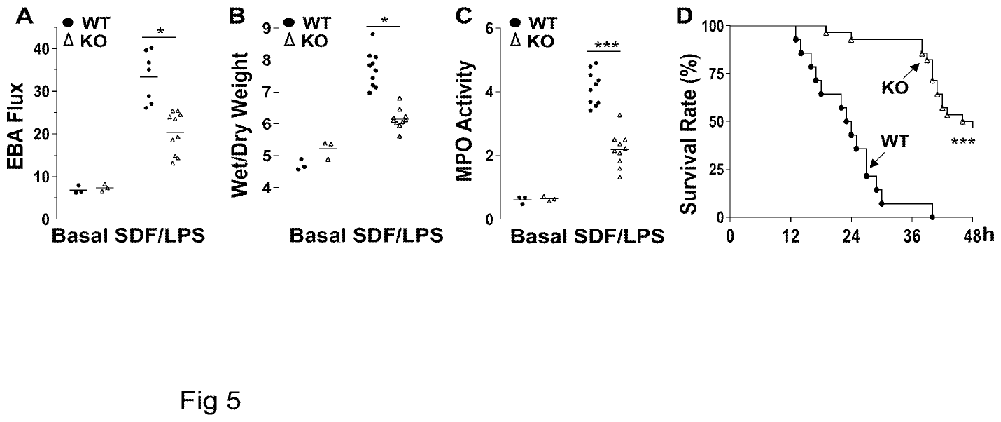

As discussed above, though the inventor discovered that signaling downstream of CXCR4 was priming EC for pyroptosis as a result of elevated SDF1, the precise signaling pathways that were potentiating this effect were not known. Therefore, the inventor demonstrated that priming-induced increases of lung injury and mortality following LPS challenge were markedly decreased in Pik3g^{-/-} (a critical catalytic component of the PI3K signaling pathway) mice compared to WT mice. See, for example, FIG. 5 Therefore, in some embodiments of the disclosed methods, the subject with elevated plasma SDF1 level at admission to ICU or early onset of ARDS is treated by administering to the subject an inhibitor of p110γ phosphoinositide 3-kinase (PI3K). Suitable inhibitors of p110γ/PI3K may include, but are not limited to, IPI-549/Eganelisib, Duvelisib, RP6530, RP6503, TG100-115 and Voxtalisib. In addition, other methods of inhibiting PI3K include, e.g., administering to the subject one or more antisense oligos that transiently inhibit expression of PI3K, for example, p110γ PI3K.

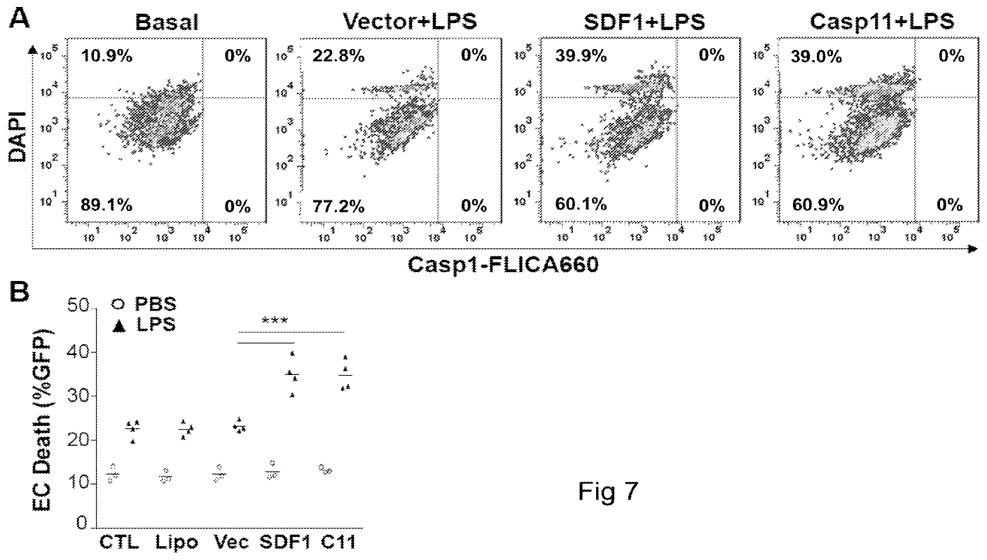

The inventors further discovered that there was a marked increase of Casp1-independent EC death in Casp11 plasmid-transduced mice following LPS challenge. See, for example, FIG. 7. The human homologs of caspase 11 are caspase 4 and caspase 5. Therefore, in some embodiments of the disclosed methods, the subject with elevated plasma SDF1 level at admission to ICU or early onset of ARDS is treated by administering to the subject an inhibitor of Caspase 4/5. Suitable inhibitors of Caspase 4/5 may include, but are not limited to, LEVD-fmk and Emricasan. Suitable inhibitors of Caspase 4/5 may include, but are not limited to siRNAs, antisense oligoes, CRISPR/guide RNAs that inhibit expression of Caspase 4/5, and dominant negative Caspase 4/5.

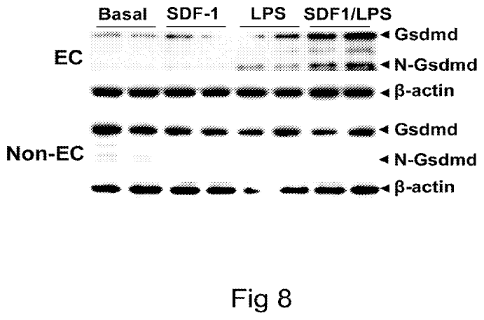

Caspases 4 and 5 are known to cleave gasdermin D (GSDMD), which can result in swelling and lysis of affected cells. The inventor demonstrated that GSDMD was cleaved in lung ECs from recombinant SDF1 (rSDF1)-primed mice following LPS challenge. See, for example, FIG. 8. Therefore, in some embodiments of the disclosed methods, the subject with elevated plasma SDF1 level at admission to ICU or early onset of ARDS is treated by administering to the subject an inhibitor of gasdermin D (GSDMD). Suitable inhibitors of GSDMD may include, but are not limited to, disulfiram, optionally administered with copper gluconate. Suitable inhibitors of GSDMD may include, but are not limited to siRNAs, antisense oligos, guide RNAs that inhibit expression of GSDMD, and dominant negative GSDMD.

In some embodiments of the disclosed methods, the subject with elevated plasma SDF1 level at admission to ICU or early onset of ARDS is treated by administering to the subject an inhibitor of gasdermin E (GSDME). Suitable inhibitors of GSDME may include, but are not limited to, Ac-DMPD-CMK and Ac-DMLD-CMK. Suitable inhibitors of GSDME may include, but are not limited to siRNAs, antisense oligoes, guide RNAs that inhibit expression of GSDME and dominant negative GSDME.

Use of the Changes of SDF1 Levels During Days after ICU Admission as a Biomarker to Guide Treatment of ARDS.

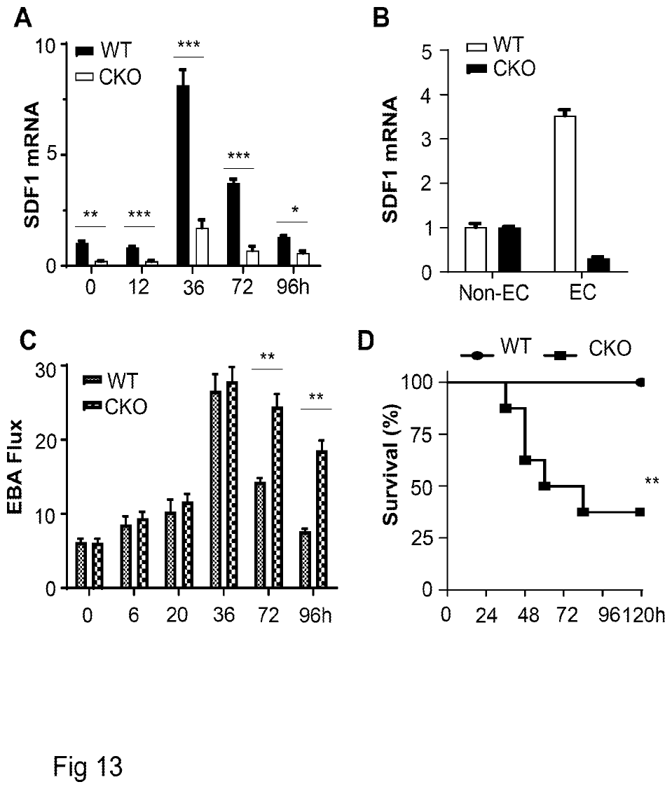

Interestingly, though the level of SDF1 is a potent predictor of disease severity and/or mortality at the time of admission of a subject to the ICU or early in the diagnosis of ARDS, the inventor further discloses that SDF1 is critical for endothelial repair following injury. For example, in mice with a conditional knockout (CKO) of SDF1, extravasation is persistently elevated following LPS challenge, indicating impaired vascular repair; in contrast extravasation returned to basal levels at 96 h post-LPS in WT mice. See, for example, FIG. 13. Therefore, in some embodiments, SDF1 is used as a biomarker to track the state of endothelial injury repair in a subject after admission to the ICU or after diagnosis of ARDS.

Importantly, the inventor discloses herein the finding that expression of SDF1 following endothelial cell (EC) injury is reduced in aged subjects compared to young subjects. Without being bound by any theory or mechanism, it is believed that the lack of expression of SDF1 in aged subjects following EC injury may explain the observation that aged subjects have increased hospitalization rate and/or mortality in response to EC injury, e.g., in response to sepsis, pneumonia, COVID-19 and associated ARDS. Thus, in some embodiment, a subject in need thereof comprises an aged individual about 65 or more years old.

Suitable biological samples for the disclosed methods may include biological samples that are isolated from the subject such as blood or blood products (e.g., plasma) and/or lung fluid (e.g., bronchoalveolar lavage fluid (BALF)). Suitable biological samples may include tissue biopsies (e.g., lung biopsies).

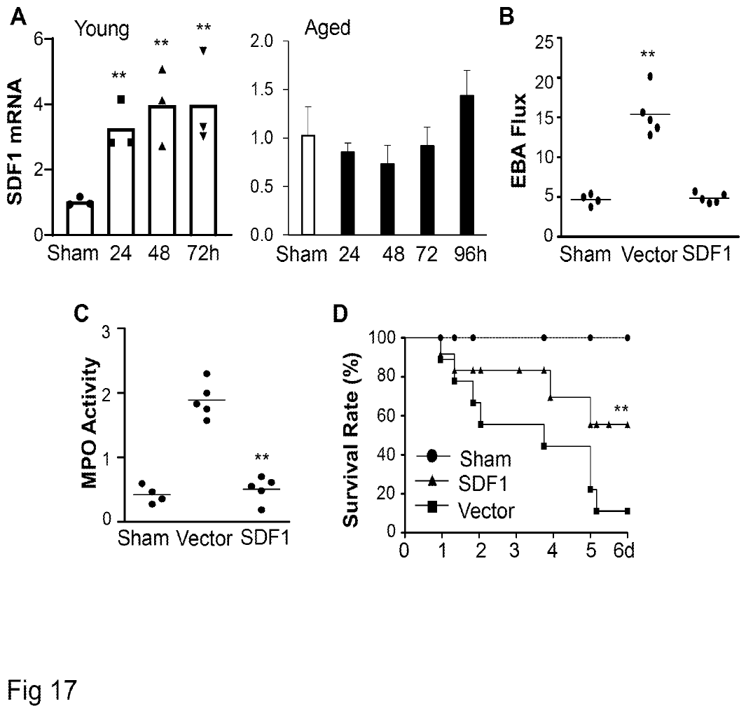

In addition to being a biomarker of endothelial repair, the inventor herein discloses that treatment of subjects with SDF1 improves endothelial repair in subjects in need thereof. For example, forced expression of SDF1 in aged mice by transduction of a plasmid encoding SDF1 normalized vascular repair and resolution of inflammation evident by basal levels of EBA flux and MPO activity at 96 h post-cecal ligation and puncture (CLP), a model of sepsis. See, for example, FIG. 17B, C. In addition, the inventor demonstrated that overexpression of SDF1 also promotes survival of aged WT mice following CLP challenge. See, for example, FIG. 17D. The disclosed methods optionally include a step of treating the subject with no marked increases of SDF1 levels in biological samples (e.g., in blood plasma) at day 2-7 compared to day 0 (at admission to ICU) or day 1. In some embodiments, the subject is treated by administering to the subject recombinant human SDF1 protein comprising the amino acid sequence of any of SEQ ID NOs:1-7 or their analogs which may comprise the properties of enhanced stability and function. The analogs may include but not limited to Ser-SDF1(S4V). Ser-SDF1(S4V) comprises an SDF1 peptide wherein the fourth amino acid is not serine, and the fifth amino acid is not leucine. In some embodiments, the subject is treated by administering to the subject small peptide SDF1 analogs. Suitable small peptide SDF1 analogs may include, but are not limited to CTCE-0324, CTCE-0214 and their analogs. In some embodiments, the subject is treated by administering to the subject viral or non-viral-mediated SDF1 cDNA or mRNA expressing the amino acid sequence of any of, e.g., SEQ ID NOs:1-7.

Exemplary viral systems for administration of SDF1 cDNA or mRNA expressing SDF1, or other similar factors, e.g., proteins with the sequence of SEQ ID NOs: 1-7, include, but are not limited to, adenoviral vectors, adeno-associated viral vectors (AAV), retroviral vectors, lentiviral vectors, etc.

Exemplary non-viral systems for administration of SDF1 cDNA or mRNA, e.g., proteins with the sequence of SEQ ID NOs: 1-7, include, but are not limited to, nanoparticles, stem cells, cell-derived microvesicles, etc.

In some embodiments of the disclosed methods, the subject is treated by administering to the subject an activator of CXCR4, or p110γ phosphoinositide 3-kinase (PI3K).

In some embodiments of the disclosed methods, the subject is elderly, e.g., age 65, 66, 67, 68, 69, 70, 71, 72, 73, 74, 75, 76, 77, 78, 79, 80, 81, 82, 83, 84, 85, 86, 88, 89, 90, 91, 92, 93, 94, 95, 96, 97, 98, 99, 100 years, or older. In some embodiments, the elderly subject has been diagnosed with ARDS.

### EXAMPLES

The following Examples are illustrative and should not be interpreted to limit the scope of the claimed subject matter.

Biomarker and Methods of Treatment of Acute Respiratory Distress Syndrome (ARDS) and severe COVID-19 and associated ARDS

Acute respiratory distress syndrome (ARDS) are complex, multi-factorial syndromes with a mortality rate as great as 30-60%. ARDS can be induced by sepsis, pneumonia, and COVID-19. Endothelial injury characterized by persistently increased lung microvascular permeability resulting in protein-rich lung edema is a hallmark of ARDS. Especially, endothelial injury is the characteristic feature of COVID-19 ARDS. However, the molecular basis of endothelial barrier disruption after sepsis, pneumonia, and COVID-19 remain poorly understood, especially, little is known about the key molecules and signaling pathways responsible for endothelial barrier dysfunction resulting in high mortality in ARDS patients. This invention defines plasma levels of stromal cell-derived factor 1 (SDF1, also known as C-X-C motif chemokine ligand 12 (CXCL12)) as a prognostic biomarker of ARDS and severe COVID-19 and associated ARDS. High levels of plasma SDF1 at admission to ICU or at the onset of ARDS induces endothelial cell pyroptosis and extensive endothelial injury which results in severe injury and great mortality in subpopulation of ARDS patients. Thus, drugs inhibiting SDF1 signaling and pyroptosis-induced endothelial injury are novel effective therapeutic agents for the subpopulation of sepsis, pneumonia and COVID-19 patients with high levels of plasma SDF1 at admission or at the onset of ARDS. These drugs include CXCR4 antagonists such as Plerixafor (AMD3100) and its analogs, p110gamma PI3K inhibitors (IPI-549/Eganelisib, Duvelisib, RP6530, RP6503, TG100-115 and Voxtalisib), Caspase 4/5 inhibitors LEVD-fmk and Emricasan, and GSDMD inhibitors such as disulfiram, and disulfiram plus copper gluconate, as well as dominant negative, antisense, siRNA, CRISPR guide RNA of Caspase 4/5 and GSDMD.

Restoration of microvessel barrier function following the injury is essential for resolving inflammatory injury and reversing lung edema. Thus, targeting microvascular leakage repair mechanisms may also represent a novel, effective therapeutic approach for treatment of ARDS. Compared to young adult patients, the incidence of ARDS resulting from sepsis, pneumonia, and COVID-19 in elderly patients (≥65 yr) and mortality rates are much higher than young adults. Our studies have demonstrated that SDF1 is markedly induced in lung ECs at the late phase of injury following sepsis challenge which is critically important to activate endothelial regeneration and vascular repair. Loss of endothelial SDF1 results in defective vascular repair and thus persistent lung injury and greater mortality. We have also observed failed induction of SDF1 in lung ECs of aged mice following sepsis challenge which is responsible for the defective vascular repair and thus persistent injury and increased morality of aged mice. Thus, drugs activating SDF1 signaling-dependent vascular repair are novel and effective therapy for subpopulation of ARDS patients and severe COVID-19 patients whose SDF1 levels in plasma or lung tissues are not markedly increased after disease diagnosis and for elderly patients with age over 65 years old. These drugs include but are not limited to recombinant human SDF1 and its analogs including Ser-SDF1(S4V), small peptide SDF1 CTCE-0324, CTCE-0214 and its analogs, viral or non-viral-mediated SDF1 cDNA or mRNA, CXCR4 activator, and p110γ phosphoinositide 3-kinase (PI3K) activator.

1. High level of plasma SDF1 at admission to ICU or at the day of diagnosis is associated with greater mortality of ARDS patients. To determine if plasma or bronchoalveolar lavage fluid (BALF) levels of SDF1 admission to ICU and at the day of diagnosis of ARDS are associated with the mortality of ARDS patients, plasma and BALF samples were collected from ARDS patients. ELISA was then carried out to quantify SDF1 levels. As shown in FIG. 1, plasma SDF1 levels in non-survival ARDS patients were significantly higher than those in survival ARDS patients which were similar to critically ill non-ARDS controls. However, BALF levels of SDF1 were not significantly different in non-survival ARDS patients compared to survival ARDS patients. This fundamental novel observation demonstrates the clinical relevance and translation potential of the findings below.

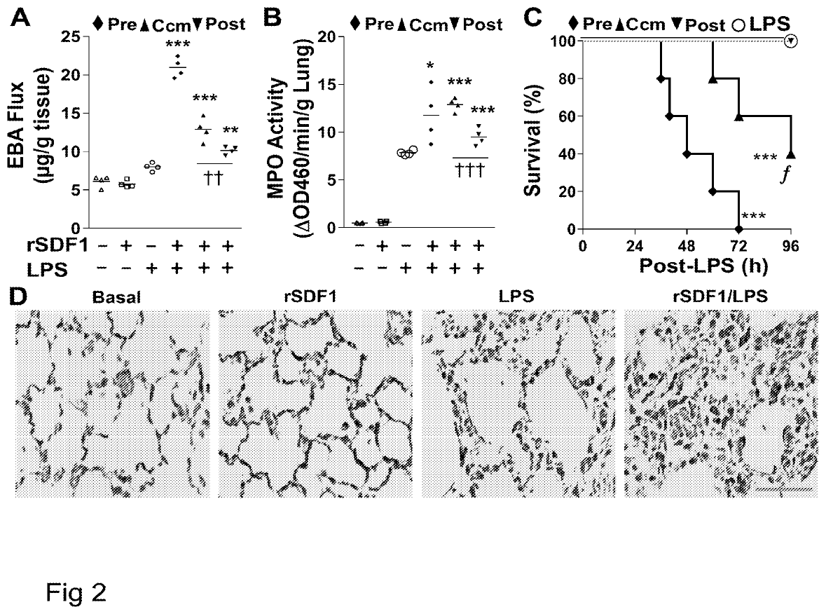

2. SDF1 priming (early recombinant SDF1α treatment or pre-expression of SDF1α in lung ECs) augments LPS-induced lung injury and mortality in mice. To determine the role of elevated circulating SDF1 level in regulating sepsis-induced lung injury and mortality, we treated C57BL/6 WT mice (3-4 month old, male and female) with recombinant SDF1α (rSDF1, i.v.) under 3 protocols (pretreatment, −8 h and 0 h; concomitant treatment, 0 h and +6 h; and post-treatment, +2 h and +8 h. 0 h=at the same time with LPS) and LPS challenge (2.5 mg/kg, i.p.). At 16 h post-LPS, lung tissues were collected for evaluation of vascular permeability by measuring extravasation of Evans blue-conjugated albumin (EBA) (22-24). rSDF1 treatment resulted in marked increases of EBA flux following LPS challenge compared to LPS alone (FIG. 2A). rSDF1 pretreatment had the most increase of EBA flux. rSDF1 treatment also augmented LPS-induced lung inflammation determined by lung myeloperoxidase (MPO) activity (FIG. 2B), a measure of neutrophil infiltration (22-24). Although SDF1 post-treatment also augment LPS-induced inflammatory lung injury, its effects were modest. SDF1 pretreatment per se didn't induce lung injury (FIG. 2A, B, D). Next, we determined the effects of SDF1 treatment on mortality following LPS challenge (6 mg/kg, i.p.). As seen in ARDS patients, both SDF1 pre- and concomitant treatments promoted LPS-induced mortality whereas SDF1 post-treatment had no effects on mortality (FIG. 2C). Consistent with EBA flux, SDF1 pretreatment induced greater mortality following LPS challenge compared to concomitant treatment (FIG. 2C). H&E staining showed increased lung neutrophil infiltration, septal thickening, and hemorrhaging in rSDF1-pretreated LPS mice (FIG. 2D).

Liposome-mediated transduction of plasmid DNA to mouse lungs has been demonstrated as an effective approach to induce transient gene expression in vivo (23, 24). We also employed a liposome-mediated gene transduction approach to express SDF1α in pulmonary vascular ECs. At 20 h prior to LPS challenge, mixture of liposome:plasmid DNA expressing SDF1α under the control of human CDH5 (encoding VE-Cadherin) promoter was administered to mice through retro-orbital injection (4 ug DNA/mouse). Empty vector DNA was administered as a control to a separate cohort mice. As shown in FIG. 3, increased expression of SDF1α in ECs also augmented LPS-induced lung injury evident by marked increases of EBA flux, lung wet/dry weight ratio, and MPO activity. Mortality was also markedly increased.

Together, these data demonstrate SDF1 priming by either elevated plasma SDF1 levels or increased expression of SDF1 in lung ECs augments LPS-induced lung injury and mortality.

3. Endothelial CXCR4 mediates SDF1 priming-induced severe lung injury and increased mortality following LPS challenge. It has been shown that both CXCR4 and CXCR7 are SDF1 receptors (25, 26). Given that CXCR4 is the predominant receptor in lung ECs, we next determined if endothelial CXCR4 mediates the priming effects of SDF1. We inactivated Cxcr4 selectively in mouse endothelium (Cxcr4^{iΔEc}) by breeding the mice with Cxcr4 floxed allele (27) with Endo-SCL-Cre-ERT2 transgenic mice containing tamoxifen-inducible Cre-ERT2 driven by the 5′ endothelial enhancer of the stem cell leukemia locus which has been shown to induce EC-restricted gene deletion (28-30). At 8 wk, littermates of Cxcr4fl/fl;Cre⁻ and Cxcr41fl/fl;Cre+ mice were treated with 2 mg tamoxifen daily for 6 days to generate “WT” mice and inducible EC-restricted Cxcr4^{iΔEC} mice, respectively. Quantitative RT-PCR showed deletion of Cxcr4 in lung ECs of Cxcr4^{iΔEC} mice but not in leukocytes (CD45⁺) (FIG. 4A). After tamoxifen treatment, littermate WT and Cxcr4^{iΔEC} mice were transduced with mixture of liposome: SDF1 plasmid to express SDF1 in lung ECs at 20 h prior to LPS challenge. Lung injury evident by EBA flux, wet/dry ratio and MPO activity was markedly attenuated in SDF1 plasmid-transduced Cxcr4^{iΔEC} mice compared to WT mice (FIG. 4B-D). Mortality in SDF1 plasmid-transduced Cxcr4^{iΔEC} mice followed LPS challenge (6 mg/kg, i.p.) was significantly reduced (FIG. 4E).

4. The GPCR-dependent p110γPI3K is the downstream signaling molecule of the SDF1 priming effects. Class I PI3K comprises the class IA (p110α, β, and δ) and class IB (p110γ, encoded by Pik3cg) isoforms. Class IA kinases (p110α, β, δ) forming a complex with SH2-containing regulatory p85-related subunits are in general activated through receptor tyrosine kinases whereas class IB, p110γ is activated by G protein-coupled receptors (GPCR) (16-18). p110β is also activated by GPCR signaling in p110γ-deficient cells (15). Given that CXCR4 is a GPCR (35), we determined the role of p110γPI3K in mediating the SDF1 priming effects employing the Pik3cg^{-/-} (p110γ knockout) mice. SDF1 priming-induced increases of lung injury and mortality following LPS challenge were markedly decreased in Pik3g^{-/-} mice compared to WT mice (FIG. 5), indicating p110γPI3K is the downstream signaling molecule mediating the SDF1 priming effects.

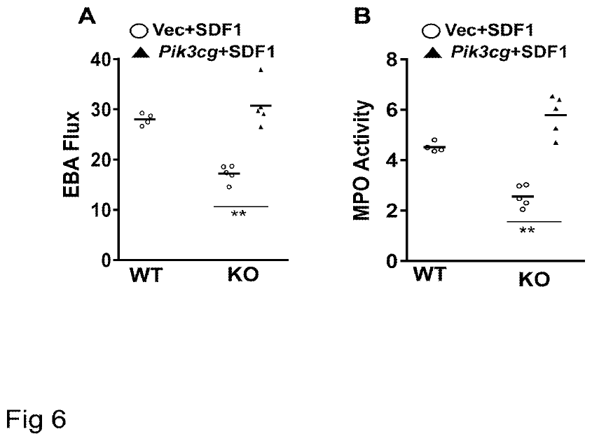

To determine if endothelial p110γPI3K mediates the SDF1 priming effects, we employed the lipoma-mediated gene transduction approach to restore p110γPI3K expression in pulmonary vascular ECs of Pik3cg mice. At 20 h prior to LPS challenge, liposome:plasmid DNA complexes expressing p110γPI3K (30 ug/mouse) or vector was administered i.v. to Pik3cg mice while WT mice were administered with vector. All mice were also transduced with SDF1 plasmid (4 ug/mouse) at the same time. EBA extravasation and MPO activity assays revealed that restored expression of endothelial p110γ in Pik3cg^{-/-} lungs normalized the SDF1 priming effects as seen in WT mice (FIG. 6A, B).

5. SDF1 priming induces marked increase of EC death following LPS challenge. To trace the changes of pulmonary ECs following sepsis challenge, we employed the tamoxifen-inducible endothelial lineage reporter mice. Mice carrying a double-fluorescent reporter expressing tdTomato (mT) prior to Cre-mediated excision and green fluorescent protein (mG) after excision with EndoSCL-Cre^{ERT2} transgenic mice to generate mTmG/EC-Cre^{ERT2} (mGFP^{iEC}) mice. 95% of lung ECs (CD45-CD31V) were labeled with GFP at 4 weeks post-tamoxifen treatment.

We next assessed EC death by FACS analysis. The mGFP^{iEC} mice were administered with mixture of liposome:plasmid DNA expressing SDF1 (CDH5 promoter) or empty vector. At 20 h post-liposome, the mice were challenged with LPS for 16 h. Isolated lung cells were subject to Casp1-FLICA660 (Immunochemistry Technologies LLC) (36) and DAPI staining followed by FACS analysis. Pre-expression of SDF1 in lung ECs induced a marked increase of Casp1-independent EC death in LPS-challenged mice whereas Casp1-dependent EC death was negligible (FIG. 7). There were no differences in non-EC death (tdTomato⁺) between Vector- and SDF1 plasmid-transduced mice.

We also observed marked increase of Casp1-independent EC death in Casp11 plasmid-transduced mice following LPS challenge as seen in SDF1 plasmid-transduced mice (FIG. 7). Casp11 is the inflammatory caspase responsible for cleavage of the pore-forming pyroptosis perforin Gsdmd leading to cell swelling and lytic cell death, i.e., pyroptosis (37-40).

6. SDF1 priming induces Gsdmd cleavage and thereby activation of pyroptosis in lung ECs. We next determined whether SDF1 priming induced marked increase of Gsdmd cleavage and activation. As shown in FIG. 8, Gsdmd was cleaved in lung ECs from rSDF1-primed mice following LPS challenge. Priming with liposome:SDF1 plasmid DNA also induced Gsdmd cleavage (data not shown). These data indicate SDF1 priming induces overwhelming EC pyroptosis following sepsis.

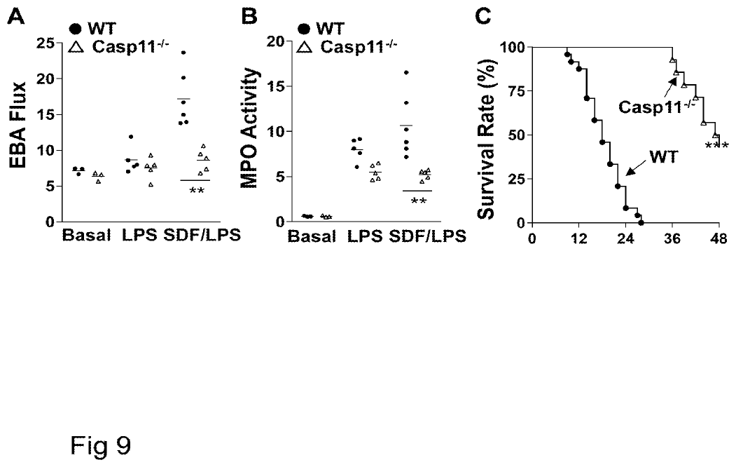

7. Caspase11 mediates SDF1 priming-augmented lung injury and mortality in LPS-challenged mice. Given that Casp11 is the inflammatory caspase responsible for Gsdmd cleavage and resultant pyroptosis, we examined the effects of SDF1 priming in Casp11^{-/-} mice. Following LPS challenge, EBA flux, lung wet/dry weight ratio, and MPO activity were markedly increased in rSDF1 primed-WT mice but not -Casp11^{-/-} mice compared to PBS-primed mice (FIG. 9A, B). The mortality rate of rSDF1-primed Casp11^{-/-} mice was markedly decreased compared to rSDF1-primed WT mice following LPS challenge (FIG. 9C).

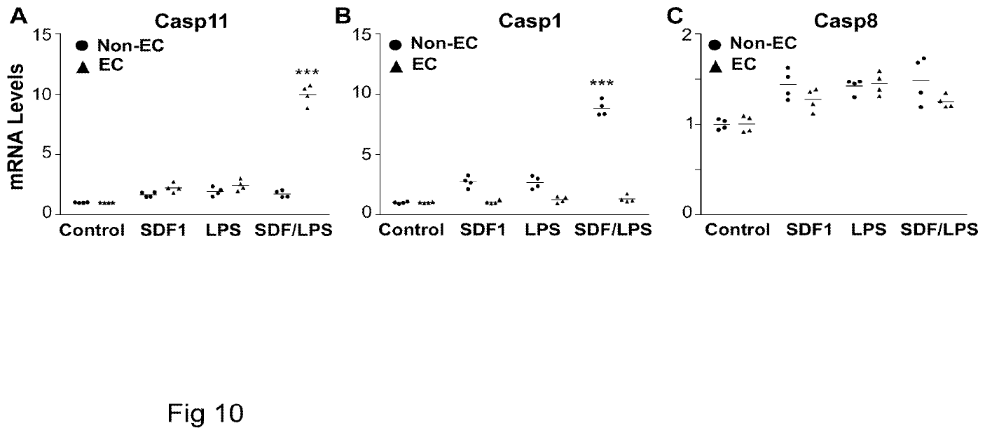

8. SDF1 priming induced expression of Casp11 but not Casp8 in lung ECs. To gain insight into the molecular mechanisms of SDF1 priming-induced EC pyroptosis, we examined expression of Casp1 and Casp11, the two inflammatory Caspases. Quantitative RT-PCR analysis showed a 10-fold increase of Casp11 expression in lung ECs but not in non-ECs of SDF1/LPS-treated WT mice compared to control (naïve) mice (FIG. 10A). Intriguingly, Casp11 expression was also increased 2-fold in lung ECs of mice treated with either SDF1 or LPS alone. In contrast, Casp1 expression was induced in non-ECs but not in ECs (FIG. 10B). Expression of Casp8, an initiator caspase of apoptosis (14) was not induced (FIG. 10C), indicating SDF1 priming selectively induces EC pyroptosis not apoptosis.

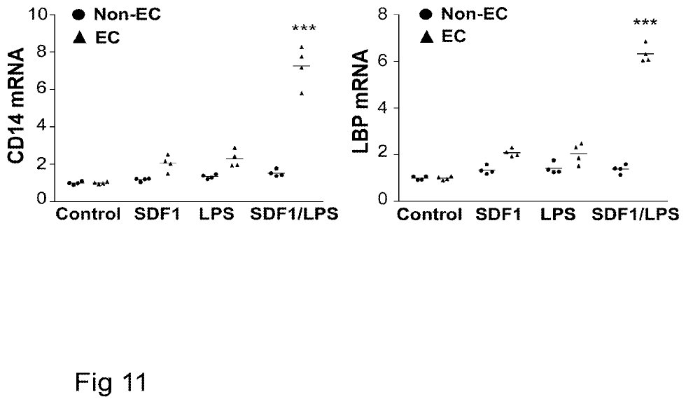

9. SDF1 priming induced expression of LPS co-receptors LBP and CD14 in lung ECs but not non-ECs, and promoted LPS binding. Quantitative RT-PCR analysis also showed marked increases (10-fold) of expression of LPS-binding protein (LBP) and CD14, which are components of the LPS receptor complex (41, 42) in lung ECs but not in non-ECs of rSDF1/LPS-treated WT mice (FIG. 11). Treatment with SDF1 or LPS alone induced only a mild increase (2-fold) of CD14 and LBP expression in lung ECs but non-ECs.

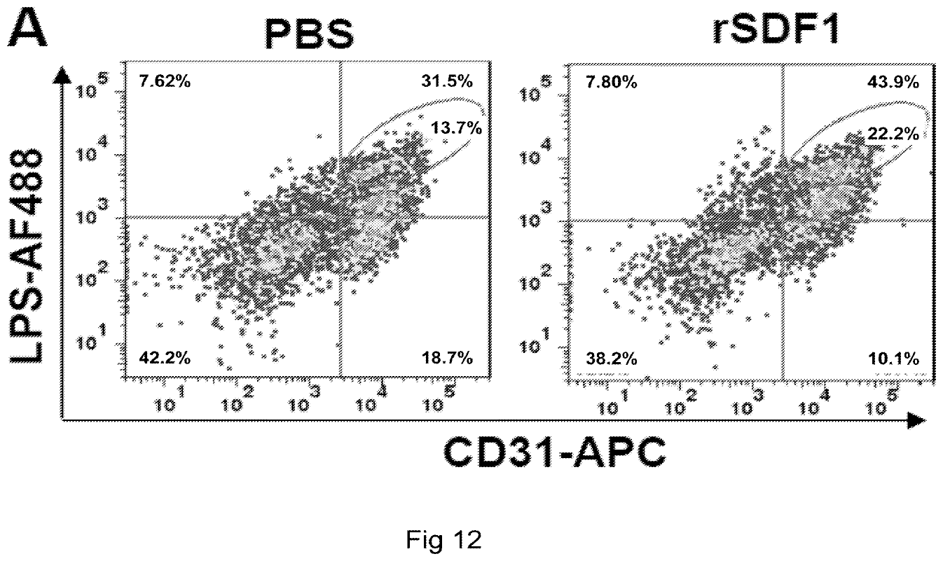

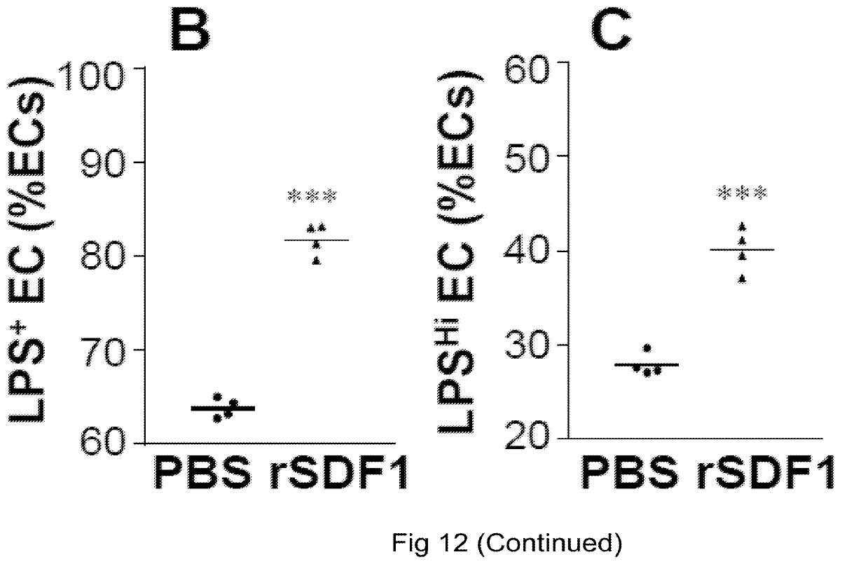

To quantify the effects of SDF1 priming on LPS binding, the mice were challenged with LPS and lung tissues were collected for cell isolation. The cells were incubated with LPS-AF488 for 30 min on ice. FACS analysis revealed marked increases of the percentages of LPS⁺ ECs and LPS^{hi} ECs in rSDF1/LPS mice compared to PBS/LPS-challenged mice. LPS⁺ non-EC population was maintained similarly at 15% of total non-ECs (CD45⁻ CD31⁻) (FIG. 12).

These studies are designed to define the molecular and cellular mechanisms of SDF1 priming in augmenting sepsis-induced lung injury and mortality and thereby provide novel therapeutic strategy for treatment of ARDS associated with high levels of plasma SDF1 at the onset of ARDS. Our data show SDF1 priming induces marked increases of expression of Casp4/5/11 leading to cleavage and activation of the pore-forming pyroptosis perforin GSDMD thus inducing excessive EC pyroptosis and severe endothelial barrier dysfunction. SDF1 priming promotes LPS internalization through increased expression of CD14 and LBP. Internalized LPS binding to Casp4/5/11 activates pyroptosis.

Summary 1-SDF1 priming induces severe endothelial injury, indicating the detrimental role of elevated plasma SDF1 at admission to ICU or early onset of ARDS in the pathogenesis of ARDS: Pyroptosis is a programmed cell death mechanism to effectively eliminate an intracellular bacteria niche and active the host through release of inflammatory mediators, while sparing uninfected neighboring cells (15-18). However, under certain pathological conditions, the potentially protective pyroptotic mechanism may be over-activated and results in excessive EC lysis and thereby severe endothelial barrier dysfunction leading to severe lung injury and increased mortality. It has been shown that TLR ligands such as LPS (TLR4), Pam3CSK4 (TLR2), and poly(I:C) (TLR3) (11) as well as oxidized phospholipids (43) can prime the cells for pyroptosis. These studies have for the first time identified SDF1 as a potent primer of EC pyroptosis. Our data have shown that SDF1 priming by i.v. injection of rSDF1 selectively induces Casp11, LBP and CD14 expression in lung ECs but not in non-ECs. Thus, SDF1 priming is unlikely to induce pyroptosis of non-ECs such as alveolar epithelial cells, which is consistent with our observation in ARDS patients that BALF SDF1 level is not associated with mortality. These studies have great translational potential as we observed that elevated circulating SDF1 level at admission to ICU or at diagnosis is associated with great mortality of ARDS patients. Thus, CXCR4 antagonists, p110gammaPI3K inhibitors, Caspase 4/5 inhibitors and GSDMD inhibitors are potential novel and effective therapeutic agents of ARDS patients with high levels of plasma SDF1 at admission to ICU after sepsis, pneumonia, and COVID-19.

11. Induced SDF1 expression in endothelial cells after sepsis-induced injury mediates vascular repair and resolution of inflammatory lung injury. The above studies demonstrate the detrimental role of elevated SDF1 before or within hours of sepsis challenge in inducing extensive endothelial cell death and injury. It is unknown if SDF1 plays a role in vascular repair after injury. We first examined SDF1 expression in mouse lungs at various times following LPS challenge. SDF1 expression was markedly induced at the late injury phase (e.g., 36 h) post-LPS challenge (FIG. 13A). To determine the role of this induced SDF1 expression in lung injury and repair, we made a mouse model with endothelial cell-specific disruption of SDF1 (CKO) (FIG. 13B). Quantitative RT-PCR analysis demonstrated blunted SDF1 induction in CKO lungs, indicating SDF1 expression is predominantly induced in ECs not non-ECs (FIG. 13A).

We next challenged the adult mice (3-4 months old) with LPS and assessed vascular permeability at various times after LPS challenge. EBA extravasation was similarly increased and peaked at 36 h post-LPS in WT and CKO mice. However, EBA extravasation is persistently elevated, indicating impaired vascular repair, in CKO lungs whereas it was returned to basal levels at 96 h post-LPS in WT mice. CKO mice also exhibited greater mortality following LPS challenge (5 mg/kg).

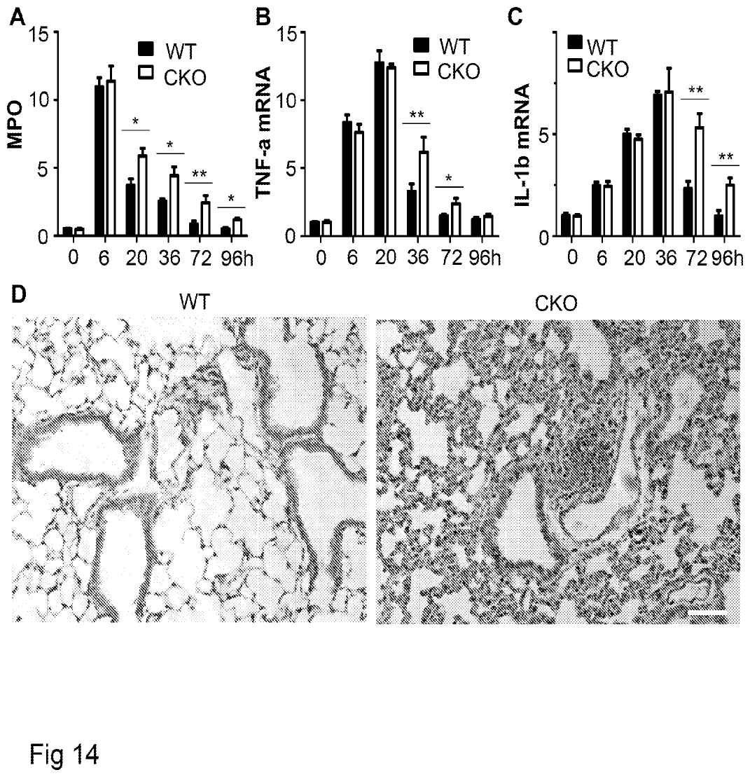

MPO activity assay demonstrate a similar increase of neutrophil infiltration in WT and CKO lungs at 6 h after LPS challenge (FIG. 14A). Lung MPO activity was decreased and return to basal levels in WT mice at 96 h post-LPS whereas it is persistently elevated in CKO mice. Expression of proinflammatory cytokines TNF-α and IL-1β was also persistently elevated in CKO lungs in contrast to WT lungs following LPS challenge (FIG. 14B, C). Histological analysis also revealed extensive inflammatory lung injury at 72 h post-LPS in CKO mice evident by extensive neutrophil sequestration and thickening of the alveolar septum (FIG. 14D). These data demonstrate impaired resolution of inflammation in CKO mice after LPS challenge.

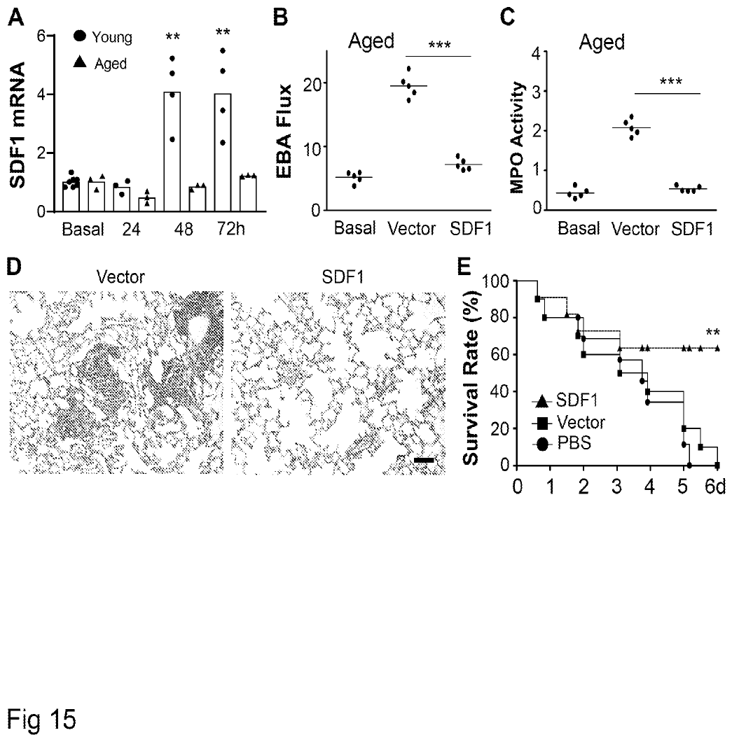

12. Impaired vascular repair in aged lungs following LPS challenge is ascribed to defective SDF1 induction. To gain insights into the mechanisms of greater incidence and mortality of ARDS in elderly patients, we investigated vascular repair in aged mice following sepsis challenge. In contrast in lungs of young adult (3-4 months) WT mice, SDF1 expression was not induced in lungs of aged WT mice after LPS challenge (FIG. 15A). EBA flux and MPO activity was persistently elevated in aged WT lungs at 72 h post-LPS challenge (FIG. 15B, C, Vector group). Histology also revealed extensive neutrophil sequestration and lung injury (FIG. 15D). To determine if inhibited SDF1 expression is responsible for the impaired vascular repair and resolution of inflammation in aged lungs, mixture of liposome:plasmid DNA expressing SDF1 under the control of CDH5 was administered to WT mice at 2 h post-LPS challenge. Vector DNA without SDF1 cDNA was administered to another cohort of mice as control. AT 72 h post-LSP, EBA flux and MPO activity were returned to levels close to basal in SDF1-transduced mice. H & E staining also shows resolved lung inflammation in SDF1-transduced mice. SDF1-transduced aged WT mice also exhibited greater survival rate following LPS challenge (5 mg/kg, i.p.) with more than 60% mice survived at 6 days post-LPS whereas all aged WT mice treated with either PBS or empty vector DNA died in the same period (FIG. 15E).

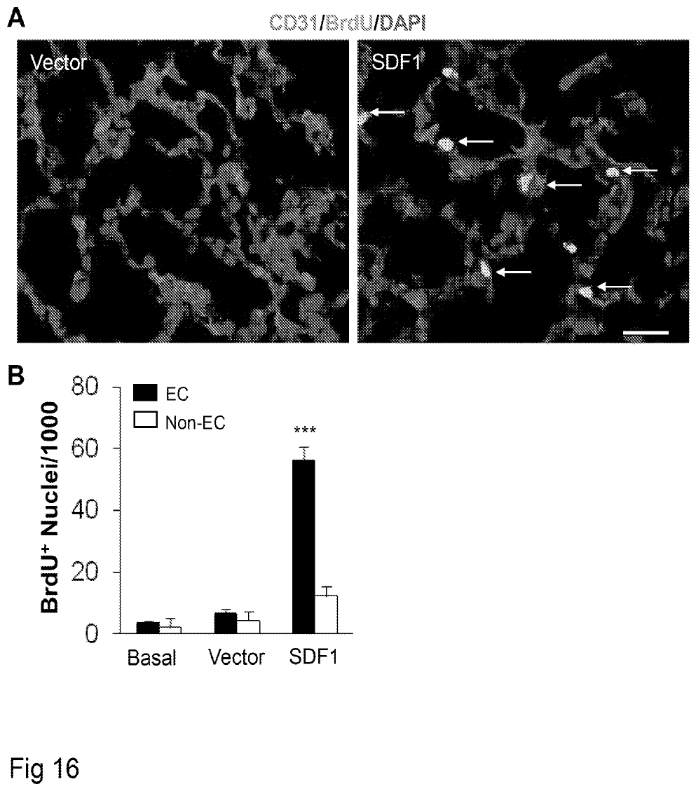

Quantification of BrdU⁺ cells shows marked induction of lung endothelial proliferation in SDF1-transduced aged WT mice in contrast to vector DNA-transduced aged WT mice (FIG. 16), suggesting restored SDF1 expression in lung ECs reactivated the dormant endothelial regeneration and thus vascular repair in aged lungs.

13. Forced SDF1 expression in aged mice reactivates vascular repair and promotes survival following polymicrobial sepsis. We also determined the role of SDF1 in reactivating vascular repair in aged mice in a clinically relevant polymicrobial sepsis model. SDF1 expression was markedly induced in lungs of young adult mice following CLP challenge but not in aged lungs (FIG. 17A). As seen the endotoxiemia model induced by LPS challenge, EBA flux and MPO activity was persistently elevated in aged WT lungs transduced with empty vector DNA at 96 h post-CLP. However, forced expression of SDF1 in aged mice by transduction of SDF1 plasmid normalized vascular repair and resolution of inflammation evident by basal levels of EBA flux and MPO activity at 96 h post-CLP (FIG. 17B, C). Overexpression of SDF1 also promote survival of aged WT mice following CLP challenge (FIG. 17D).

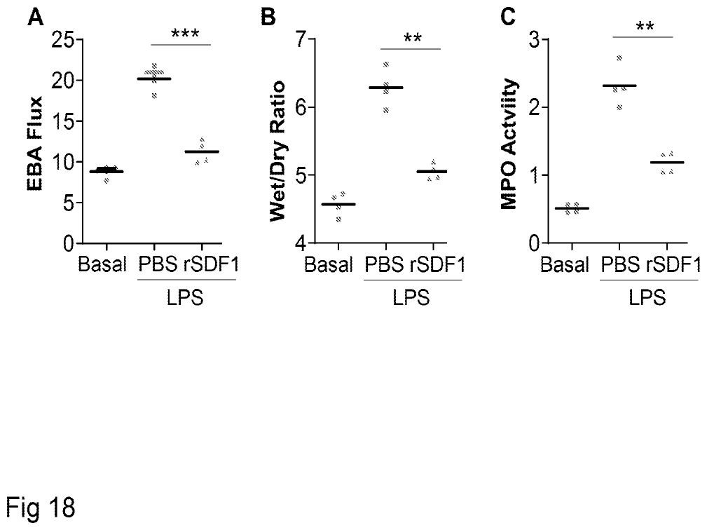

14. Therapeutic treatment of recombinant human SDF1α reactivates lung vascular repair and resolution of inflammation in aged mice. To determine if recombinant human SDF1α can be used as a therapy to reactivate vascular repair and resolution of inflammation in elderly ARDS patients, aged WT mice were treated with either recombinant human SDF1α or PBS at 16 h post-LPS challenge. At 72 h post-LPS, lung tissues were collected for assessment of EBA and MPO activity as well as wet/dry weight ratio (indicative of lung edema). Elevated lung EBA flux seen in PBS-treated mice was markedly reduced in rSDF1-treated mice which was close to basal levels (FIG. 18A). Consistently, lung edema was markedly inhibited in rSDF1-treated mice (FIG. 18B). rSDF1 treatment also markedly reduced MPO activity (FIG. 18C). Together, these data demonstrate recombinant human SDF1 is an effective therapy for elderly ARDS patients.

15. Summary 2: Recombinant human SDF1 is an effective therapy for ARDS in elderly patients and patients with blunted plasma or lung SDF1 expression. These data provide unequivocal evidence that SDF1 induced after sepsis challenge plays an important role in mediating vascular repair and resolution of inflammation and thus survival. Blunted SDF1 expression is responsible for defective vascular repair and increased mortality in aged mice. Recombinant human SDF1 is an effective therapy for ARDS in elderly patients. Inhibited increase of plasma SDF1 or lung SDF1 after injury induced by sepsis, pneumonia and COVID-19 results in defective vascular repair leading to severe ARDS and high mortality.

### REFERENCES

1. Rubenfeld, G. D., Caldwell, E., Peabody, E., Weaver, J., Martin, D. P., Neff, M., Stern, E. J., and Hudson, L. D. 2005. Incidence and outcomes of acute lung injury. N Engl J Med 353:1685-1693.
2. Matthay, M. A., Ware, L. B., and Zimmerman, G. A. 2012. The acute respiratory distress syndrome. J Clin Invest 122:2731-2740.
3. Matthay, M. A., Zemans, R. L., Zimmerman, G. A., Arabi, Y. M., Beitler, J. R., Mercat, A., Herridge, M., Randolph, A. G., Calfee C S. 2019. Acute respiratory distress syndrome. Nat Rev Dis Primers 5:18.
4. Marini, J. J., and Gattinoni, L. 2020. Management of COVID-19 respiratory distress. JAMA, 323: 2329-2330.
5. Huertas, A., Montani, D., Savale, L., Pichon, J., Tu, L., Parent, F., Guignabert, C., and Humbert, M. 2020. Endothelial cell dysfunction: a major player in SARS-CoV-2 infection (COVID-19)? Eur Respir J. 56: 2001634.
6. Mangalmurti, N. S., Reilly, J. P., Cines, D. B., Meyer, N.J., Hunter, C. A., and Vaughan A. E. 2020. COVID-19-associated acute respiratory distress syndrome clarified: A vascualr endotype? Am J Respir Crit Care Med. 202:750-753.
7. Mirza, M. K., Sun, Y., Zhao, Y. D., Potula, H. H., Frey, R. S., Vogel, S. M., Malik, A. B., and Zhao, Y. Y. 2010. FoxM1 regulates re-annealing of endothelial adherens junctions through transcriptional control of beta-catenin expression. J Exp Med 207:1675-1685. PMCID: PMC2916140
8. Bhattacharya, J., and Matthay, M. A. 2013. Regulation and repair of the alveolar-capillary barrier in acute lung injury. Annu Rev Physiol 75:593-615.
9. Mehta, D., and Malik, A. B. 2006. Signaling mechanisms regulating endothelial permeability. Physiol Rev 86:279-367.
10. Wallach, D., Kang, T. B., Dillon, C. P., and Green, D. R. 2016. Programmed necrosis in inflammation: Toward identification of the effector molecules. Science 352: aaf2154.
11. Cheng, K. T., Xiong, S., Ye, Z., Hong, Z., Di, A., Tsang, K. M., Gao, X., An, S., Mittal, M., Vogel, S. M., et al. 2017. Caspase-11-mediated endothelial pyroptosis underlies endotoxemia-induced lung injury. J Clin Invest 127:4124-4135.
12. Kayagaki, N., Wong, M. T., Stowe, I. B., Ramani, S. R., Gonzalez, L. C., Akashi-Takamura, S., Miyake, K., Zhang, J., Lee, W. P., Muszynski, A., et al. 2013. Noncanonical inflammasome activation by intracellular LPS independent of TLR4. Science 341:1246-1249.
13. Hagar, J. A., Powell, D. A., Aachoui, Y., Ernst, R. K., and Miao, E. A. 2013. Cytoplasmic LPS activates caspase-11: implications in TLR4-independent endotoxic shock. Science 341:1250-1253.
14. Fink, S. L., and Cookson, B. T. 2005. Apoptosis, pyroptosis, and necrosis: mechanistic description of dead and dying eukaryotic cells. Infect Immun 73:1907-1916.
15. Jorgensen, I., and Miao, E. A. 2015. Pyroptotic cell death defends against intracellular pathogens. Immunol Rev 265:130-142.
16. Aachoui, Y., Leaf, I. A., Hagar, J. A., Fontana, M. F., Campos, C. G., Zak, D. E., Tan, M. H., Cotter, P. A., Vance, R. E., Aderem, A., et al. 2013. Caspase-11 protects against bacteria that escape the vacuole. Science 339:975-978.
17. Maltez, V. I., Tubbs, A. L., Cook, K. D., Aachoui, Y., Falcone, E. L., Holland, S. M., Whitmire, J. K., and Miao, E. A. 2015. Inflammasomes Coordinate Pyroptosis and Natural Killer Cell Cytotoxicity to Clear Infection by a Ubiquitous Environmental Bacterium. Immunity 43:987-997.
18. Jorgensen, I., Zhang, Y., Krantz, B. A., and Miao, E. A. 2016. Pyroptosis triggers pore-induced intracellular traps (PITs) that capture bacteria and lead to their clearance by efferocytosis. J Exp Med 213:2113-2128.
19. Kayagaki, N., Stowe, I. B., Lee, B. L., O'Rourke, K., Anderson, K., Warming, S., Cuellar, T., Haley, B., Roose-Girma, M., Phung, Q. T., et al. 2015. Caspase-11 cleaves gasdermin D for non-canonical inflammasome signalling. Nature 526:666-671.
20. Shi, J., Zhao, Y., Wang, K., Shi, X., Wang, Y., Huang, H., Zhuang, Y., Cai, T., Wang, F., and Shao, F. 2015. Cleavage of GSDMD by inflammatory caspases determines pyroptotic cell death. Nature 526:660-665.
21. Liu, X., Zhang, Z., Ruan, J., Pan, Y., Magupalli, V. G., Wu, H., and Lieberman, J. 2016. Inflammasome-activated gasdermin D causes pyroptosis by forming membrane pores. Nature 535:153-158.
22. Zhao, Y. D., Huang, X., Yi, F., Dai, Z., Qian, Z., Tiruppathi, C., Tran, K., and Zhao, Y. Y. 2014. Endothelial FoxM1 mediates bone marrow progenitor cell-induced vascular repair and resolution of inflammation following inflammatory lung injury. Stem Cells 32:1855-1864. PMCID: PMC4383792.
23. Huang, X., Dai, Z., Cai, L., Sun, K., Cho, J., Albertine, K. H., Malik, A. B., Schraufnagel, D. E., and Zhao, Y. Y. 2016. Endothelial p1 l0gammaPI3K Mediates Endothelial Regeneration and Vascular Repair After Inflammatory Vascular Injury. Circulation 133:1093-1103. PMCID: PMC4792690.
24. Huang, X., Zhang X, Zhao D X, Yin J, Hu G, Evans C E and Zhao, Y. Y. 2019. Endothelial hypoxia-inducible factor-la is required for vascular repair and resolution of inflammatory lung injury through FoxM1. Am J Pathol 2019 May 20. pii: S0002-9440(18)31116-7.
25. Burns, J. M., Summers, B. C., Wang, Y., Melikian, A., Berahovich, R., Miao, Z., Penfold, M. E., Sunshine, M. J., Littman, D. R., Kuo, C. J., et al. 2006. A novel chemokine receptor for SDF1 and I-TAC involved in cell survival, cell adhesion, and tumor development. J Exp Med 203:2201-2213.
26. Costello, C. M., McCullagh, B., Howell, K., Sands, M., Belperio, J. A., Keane, M. P., Gaine, S., and McLoughlin, P. 2012. A role for the CXCL12 receptor, CXCR7, in the pathogenesis of human pulmonarty vascualr disease. Eur Respir J 39:1415-24.
27. Schober, A., Karshovska, E., Zernecke, A., and Weber, C. 2006. SDFlalpha-mediated tissue repair by stem cells: a promising tool in cardiovascular medicine? Trends Cardiovasc Med 16:103-108.
28. Muzumdar, M. D., Tasic, B., Miyamichi, K., Li, L., and Luo, L. 2007. A global double-fluorescent Cre reporter mouse. Genesis 45:593-605.
29. Gothert, J. R., Gustin, S. E., van Eekelen, J. A., Schmidt, U., Hall, M. A., Jane, S. M., Green, A. R., Gottgens, B., Izon, D. J., and Begley, C. G. 2004. Genetically tagging endothelial cells in vivo: bone marrow-derived cells do not contribute to tumor endothelium. Blood 104:1769-1777.
30. Tran, K. A., Zhang, X., Predescu, D., Huang, X., Machado, R. F., Gothert, J. R., Malik, A. B., Valyi-Nagy, T., and Zhao, Y. Y. 2016. Endothelial beta-Catenin Signaling Is Required for Maintaining Adult Blood-Brain Barrier Integrity and Central Nervous System Homeostasis. Circulation 133:177-186. PMCID: PMC4814374.
31. Fruman, D. A., and Cantley, L. C. 2002. Phosphoinositide 3-kinase in immunological systems. Semin Immunol 14:7-18.
32. Leevers, S. J., Vanhaesebroeck, B., and Waterfield, M. D. 1999. Signalling through phosphoinositide 3-kinases: the lipids take centre stage. Curr Opin Cell Biol 11:219-225.
33. Stephens, L. R., Eguinoa, A., Erdjument-Bromage, H., Lui, M., Cooke, F., Coadwell, J., Smrcka, A. S., Thelen, M., Cadwallader, K., Tempst, P., et al. 1997. The G beta gamma sensitivity of a PI3K is dependent upon a tightly associated adaptor, p101. Cell 89:105-114.
34. Guillermet-Guibert, J., Bjorklof, K., Salpekar, A., Gonella, C., Ramadani, F., Bilancio, A., Meek, S., Smith, A. J., Okkenhaug, K., and Vanhaesebroeck, B. 2008. The p110beta isoform of phosphoinositide 3-kinase signals downstream of G protein-coupled receptors and is functionally redundant with p110gamma. Proc Natl Acad Sci USA 105:8292-8297.
35. Wescott, M. P., Kufareva, I., Paes, C., Goodman, J. R., Thaker, Y., Puffer, B. A., Berdougo, E., Rucker, J. B., Handel, T. M., and Doranz, B. J. 2016. Signal transmission through the CXC chemokine receptor 4 (CXCR4) transmembrane helices. Proc Natl Acad Sci USA 113:9928-9933.
36. Chapman, K. 1992. Synthesis of a potent, reversible inhibitor of interleukin-1l converting enzyme. Bioorganic & Medicinal Chemistry Letters 2:613-618.
37. Ding, J., Wang, K., Liu, W., She, Y., Sun, Q., Shi, J., Sun, H., Wang, D. C., and Shao, F. 2016. Pore-forming activity and structural autoinhibition of the gasdermin family. Nature 535:111-116.
38. Sborgi, L., Ravotti, F., Dandey, V. P., Dick, M. S., Mazur, A., Reckel, S., Chami, M., Scherer, S., Huber, M., Bockmann, A., et al. 2015. Structure and assembly of the mouse ASC inflammasome by combined NMR spectroscopy and cryo-electron microscopy. Proc Natl Acad Sci USA 112:13237-13242.
39. Chen, X., He, W. T., Hu, L., Li, J., Fang, Y., Wang, X., Xu, X., Wang, Z., Huang, K., and Han, J. 2016. Pyroptosis is driven by non-selective gasdermin-D pore and its morphology is different from MLKL channel-mediated necroptosis. Cell Res 26:1007-1020.
40. Russo, H. M., Rathkey, J., Boyd-Tressler, A., Katsnelson, M. A., Abbott, D. W., and Dubyak, G. R. 2016. Active Caspase-1 Induces Plasma Membrane Pores That Precede Pyroptotic Lysis and Are Blocked by Lanthanides. J Immunol 197:1353-1367.
41. Ranoa, D. R., Kelley, S. L., and Tapping, R. I. 2013. Human lipopolysaccharide-binding protein (LBP) and CD14 independently deliver triacylated lipoproteins to Toll-like receptor 1 (TLR1) and TLR2 and enhance formation of the ternary signaling complex. J Biol Chem 288:9729-9741.
42. Zanoni, I., Ostuni, R., Marek, L. R., Barresi, S., Barbalat, R., Barton, G. M., Granucci, F., and Kagan, J. C. 2011. CD14 controls the LPS-induced endocytosis of Toll-like receptor 4. Cell 147:868-880.
43. Zanoni, I., Tan, Y., Di Gioia, M., Broggi, A., Ruan, J., Shi, J., Donado, C. A., Shao, F., Wu, H., Springstead, J. R., et al. 2016. An endogenous caspase-11 ligand elicits interleukin-1 release from living dendritic cells. Science 352:1232-1236.

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

1. A method comprising:
(a) detecting an expression level of stromal cell-derived factor 1 (SDF1) in a biological sample from a subject having or suspected of having acute respiratory distress syndrome (ARDS); and optionally
(b) treating the subject for ARDS and severe COVID-19 and associated ARDS.

2. The method of claim 1, wherein detecting an expression level comprises detecting a concentration of SDF1 protein in the biological sample.

3. The method of claim 1 or 2, wherein detecting an expression level comprises detecting a concentration of SDF1 protein in the biological sample, wherein the protein comprises an amino acid sequence selected from the group consisting of SEQ ID NO: 1-7.

4. The method of claim 2 or 3, further comprising providing a reference concentration of SDF1 and wherein, if the detected concentration of SDF1 protein in the biological sample is equal to or higher than a reference concentration, then the subject is treated for ARDS or severe COVID-19 and associated ARDS.

5. The method of claim 4, wherein the biological sample is obtained from the subject at about the time of admission of the subject to intensive care unit (ICU).

6. The method of claim 4 or 5, wherein the biological sample is obtained from the subject at about the onset of ARDS.

7. The method of claim 5, wherein the admission to ICU is due to sepsis, pneumonia, or COVID-19.

8. The method of claims 1-7, wherein the detected plasma concentration and/or reference concentration is at least about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, or higher.

9. The method of any of claims 1-8, wherein the biological sample is selected from plasma, serum, or blood.

10. The method of any of the foregoing claims, wherein treating the subject comprises administering to the subject an antagonist of C-X-C motif chemokine receptor 4 (CXCR4).

11. The method of claim 10, wherein treating the subject comprises administering to the subject a therapeutic agent selected from the group consisting of Plerixafor (AMD3100), CTCE-9908 and their analogs.

12. The method of claim 11, wherein the subject is treated for no more than about 3 days.

13. The method of any of claims 1-9, wherein treating the subject comprises administering to the subject an inhibitor of p110γ phosphoinositide 3-kinase (PI3K) from the group consisting of IPI-549/Eganelisib, Duvelisib, RP6530, RP6503, TG100-115 and Voxtalisib, and their analogs or other p110γPI3k inhibitors or PI3K inhibitors.

14. The method of claim 10 or 13, wherein treating the subject comprises administering to the subject an siRNA, shRNA, one or more antisense oligos that transiently inhibit expression of CXCR4, or one or more antisense oligos that transiently inhibit expression of p110γPI3K.

15. The method of claim 13 or 14, wherein the subject is treated for no more than about 3 days.

16. The method of any of claims 1-9, wherein treating the subject comprises administering to the subject an inhibitor of Caspase 4/5.

17. The method of claim 15, wherein treating the subject comprises administering to the subject a therapeutic agent selected from the group consisting of LEVD-fmk and Emricasan and their analogs.

18. The method of claim 15, wherein treating the subject comprises administering to the subject an siRNA, shRNA, an antisense oligo, a CRISPR/guide RNA, or a genome editing system that inhibits expression of Caspase 4/5, or a dominant negative Caspase 4/5 that inhibits Caspase 4/5 function.

19. The method of any of claims 1-9, wherein treating the subject comprises administering to the subject an inhibitor of gasdermin D (GSDMD) or gasdermin E (GSDME).

20. The method of claim 19, wherein treating the subject comprises administering to the subject disulfiram optionally with copper gluconate, or disulfiram analogs.

21. The method of any of claims 1-9, wherein treating the subject comprises administering to the subject an siRNA, shRNA, an antisense oligo, a CRISPR/guide RNA, or a genome editing system that inhibits expression of GSDMD or GSDME.

22. The method of any of claims 1-9, wherein treating the subject comprises administering to the subject one or both of (1) a dominant negative GSDMD that inhibits GSDMD function, and (2) a dominant negative GSDME that inhibits GSDME function.

23. A method comprising administering treatment for acute respiratory distress syndrome (ARDS) or severe COVID-19 to a subject exhibiting a concentration of stromal cell-derived factor 1 (SDF1) protein in the blood (plasma, serum) sample from the subject at admission to ICU or early onset of ARDS that is at least about 1 ng/ml, 2 ng/ml, 3 ng/ml, 4 ng/ml, 5 ng/ml, 6 ng/ml, 7 ng/ml, 8 ng/ml, 9 ng/ml, 10 ng/ml, 11 ng/ml, 12 ng/ml, 13 ng/ml, 14 ng/ml, 15 ng/ml, or higher.

24. The method of claim 23, wherein the treatment comprises administering to the subject an antagonist of C-X-C motif chemokine receptor 4 (CXCR4).

25. The method of claim 24, wherein the treatment comprises administering to the subject a therapeutic agent selected from the group consisting of Plerixafor (AMD3100) and CTCE-9908 and their analogs.

26. The method of claim 23, wherein the treatment comprises administering to the subject an inhibitor of p110γ phosphoinositide 3-kinase (PI3K).

27. The method of claim 26, wherein the treatment comprises administering to the subject a therapeutic agent selected from the group consisting of IPI-549/Eganelisib, Duvelisib, RP6530, RP6503, TG100-115 and Voxtalisib and their analogs, or other p110γPI3k inhibitors, or PI3K inhibitors.

28. The method of claim 23, wherein treating the subject comprises administering to the subject an siRNA, shRNA, one or more antisense oligos that transiently inhibit expression of CXCR4 or one or more antisense oligos that transiently inhibit expression of p110γPI3K.

29. The method of claim 23, wherein the treatment comprises administering to the subject an inhibitor of Caspase 4/5.

30. The method of claim 29, wherein the treatment comprises administering to the subject a therapeutic agent selected from the group consisting of LEVD-fmk and Emricasan and their analogs.

31. The method of claim 23, wherein the treatment comprises administering to the subject a siRNA, shRNA, an antisense oligo, a CRISPR/guide RNA, a genome editing system that inhibits expression of Caspase 4/5, or a dominant-negative Caspase 4/5 that inhibits Caspase 4/5 function.

32. The method of claim 23, wherein the treatment comprises administering to the subject an inhibitor of gasdermin D (GSDMD) or gasdermin E (GSDME).

33. The method of claim 32, wherein the treatment comprises administering to the subject disulfiram optionally with copper gluconate, or disulfiram analogs.

34. The method of claim 32, wherein the treatment comprises administering to the subject a siRNA, shRNA, an antisense oligo, a CRISPR/guide RNA, or a genome editing system that inhibits expression of GSDMD or GSMDE.

35. The method of claim 32, wherein treating the subject comprises administering to the subject a dominant negative GSDMD that inhibits GSDMD function.

36. The method of any of claims 1 and 2, which comprise diagnosing the subject having acute respiratory distress syndrome (ARDS) or severe COVID-19 and associated ARDS for treatment 1, 2, 3, 4, 5, or 7 days after ICU admission.

37. The method of claim 36 comprising detecting an expression level of stromal cell-derived factor 1 (SDF1) in a biological sample (blood, plasma, serum, lung biopsy) from a subject having ARDS, or severe COVID-19 and associated with ARDS in a series of days following ICU admission (day 0), e.g. day 1, day 2, day 3, day 4, day 5 and day 7.

38. The method of any claim 36 or 37, wherein if no marked increases (at least 50%) of SDF1 levels in any one or more days from day 2 to day 7 compared to day 0 or day 1 are observed, the subject will be treated with SDF1 or activators of SDF1 at day 3 or after.

39. The method of claim 38, wherein treating the subject comprises administering to the subject a recombinant human SDF1.

40. The method of claim 35, wherein the recombinant human SDF1 comprises a protein with the amino acid sequence of any of SEQ ID NOs: 1-7, Ser-SDF1(S4V), or their analogues.

41. The method of claim 38, wherein treating the subject comprises administering to the subject a SDF1 small peptide from the group of CTCE-0214, CTCE-0324, and their analogs.

42. The method of claim 38, wherein treating the subject comprises administering to the subject SDF1 comprising a protein comprising the amino acid sequence of any SEQ ID NOs:1-7, Ser-SDF1(S4V), or their analogs using a viral vector, a non-viral vector, cell, stem cells, mesenchymal stem cells, or microvesicles from the cells as a carrier.

43. The method of claim 42, wherein the non-viral vector is a nanoparticle or a liposome.

44. The method of claim 38, wherein treating the subject comprises administering to the subject a CXCR4 activator.

45. The method of claim 38, wherein treating the subject comprises administering to the subject a p110gamma PI3K activator.

46. The method of any of the foregoing claims, wherein the subject is an elderly patient, e.g., at age of 65, 70, 75, 80 years or older.
